SEQUENCE LISTING (41 sequences)

SEQ ID NO: 1 - DNA, 2980 bp, Prunus persica
tctccccagtatcgctttgcaatccgatacaatatttatatggataaaaatgtaatttagtgttattatatgaaatttagcaatgataattaagagaaattaaaagggaaaataaaggagaaaaatggaggcccatcaactccattaagagacagaaaaaaagagtgggcaagaaagcaaatgagtcatcagtttcagacttcttcttactttctctctctttaagctctgaaacaaatgtagagaatttataccctcaaaattgtcactcaccttccgcacggtaagtcccaatccagctttctctaactttctgtttggtctcccagaaaatacaacgaacaaaagaaaaagaaaaaaaaacgaagaaaattcggatcgcagtaactgccactagtcttagaccaagctgttatttatttattttattttttgtgaaattttatttttaagctctttactctttgtgtcgtctctgtgactgagagtgtgtttgggttgtgaatgatttgaaggtcttgaaaaggcgttatgatgagggagatgagcaaagaacacccaccagagcctcttgatttcttcatttggactgttgaggtaaccaattttcagtctctttttctctttttctgtccttgtttttggatatttctcattcaatcaagtagatcttgtgcgttgttactggagaattgttgtttgggacatctgattttgacttcattttttttgttaatttattgctagctcatacatctgtttctctttgagttaggctttgtatatatgtgtgtttgtatgtattaactttttgaaattttggggttttttttaacatcttgtgatgtattttctttttgatggggttgttactgggggaacagttatcaagtggtaggttttagttttgattattgtgaaatagaccaacttaatgaatcgaaatagaggagacttttcatgtaatctgctaattatctttcatacttggcaagctgggaaattcatcagtttcatttgaatccatctcatgttgttactacaggaaataattgccttcactcatcaattcatcctcctccaactattattcgacacaatcttctttctttgagttatggttcttgaggtgtcaggcgtttgactctttgtcactttataaaacgattctaatatgatacagaacaatcgtttgttgttagtgcaataccgtattatctgttgctattcccttggtcaggaccctcctcatcttggggtgtggaaattgtttccctgtccatagaatgatgaatggattataattttttttttaacaacttcacaagtgaacaaaactatgagctaaagcttgaatgtggtctttgactctgaagttgtgctaaagatgcctccacctaactattagtagctatgagtaagtacgttacagtgaaaataaagagacaaacacattactattggtcttaggaatcttaggattacctctgggaacagatattttgtgactaaaggggattattgatattaaaattaaacacagccaagttacagacaaaaacatttctttgccatgtgaagtaatgccgtttgatatggatgaactctagtcggataatctcaaaagaaagagtttaaattgccctaagtttaaggttgtcctgaacaaaaatttgggtgaatattgttttttttatttttatttttgggttggagggactgttatattataacagtgacttaataatagcctgagtttatcatgtgccctgatgaaggcctcatgtacatattttttggccatatataataaaacgaatataaagatacatggctcacccacatgtattctcatgtttattttcccctttcgtacaggatgtcggtttgtggttggaagagataaacctcggcagttaccggcaaacttttaaggaaaatggagtcaatggagaatacctggaaggcatgtcaatgtttactactgagcagattcttcggtttataagacggtgccacatgaaatggggagactttatcacgctgtgtaaggaactaaggcgaattaaaggtctgtagcctagaaattttcctagcagtctttatcatgctgtgaaaaaaatgtctttgtgttcgttcttatatcaaaaatttcatttagtctctggagttattggataaatagatttgaataagggttgtgattatcttactgttgcattttctatgcacaggtacatatgacatttgattattggaaaatataattattccaaaagtaaaacagaaaagagagtttttgtagagtatatggattggtgagatagactggccatgcatgatccattcatttcatgcactcaaatgtctactgatagcatagcacgctgacactaagaaaccgttcttatgcctcattcaatagcattgtgaaactctctcttgatctcattatctatcctagcaagctgagaatttaaacctttctggaatcatatttcccttgagctacatatatatgcctttttcctctgaaatttgaattgcatttagataccatctgtgtgtgtgctaaaaccaagtttatgtccagtggcttgcctgaaaggggagcaaaaggtccgcaggccatggtgggctccatcttgcctctcagtagtatttgtcaaggtggcaaagcgtaacagacaatcacgggttgtttccttgaagctggaaccataaaggatgatcttatatgtacctacatatattttctttatttctttttcttttttttgcctagagaacagagtaggaaagtacttttttcctttcttttttttctctcgagtctgtcaaattacatgttcgttcttctttggagaactctgtttttatatagcctttaatgtatctatcgctggtcttttgatctttatatgtttgtttactttgagaaccatgatgaggcaatcaagttcttgttgcagatcatcca

SEQ ID NO: 2 - DNA, 390 bp, Prunus persica
atgatgagggagatgagcaaagaacacccaccagagcctcttgatttcttcatttggactgttgaggatgtcggtttgtggttggaagagataaacctcggcagttaccggcaaacttttaaggaaaatggagtcaatggagaatacctggaaggcatgtcaatgtttactactgagcagattcttcggtttataagacggtgccacatgaaatggggagactttatcacgctgtgtaaggaactaaggcgaattaaagtggcttgcctgaaaggggagcaaaaggtccgcaggccatggtgggctccatcttgcctctcagtagtatttgtcaaggtggcaaagcgtaacagacaatcacgggttgtttccttgaagctggaaccataa

SEQ ID NO: 3 - protein, 129 aa, Prunus persica
MMREMSKEHPPEPLDFFIWTVEDVGLWLEEINLGSYRQTFKENGVNGEYLEGMSMFTTEQILRFIRRCHMKWGDFITLCKELRRIKVACLKGEQKVRRPWWAPSCLSVVFVKVAKRNRQSRVVSLKLEP

SEQ ID NO: 4 - DNA, 390 bp, Prunus persica
ttatggttccagcttcaaggaaacaacccgtgattgtctgttacgctttgccaccttgacaaatactactgagaggcaagatggagcccaccatggcctgcggaccttttgctcccctttcaggcaagccactttaattcgccttagttccttacacagcgtgataaagtctccccatttcatgtggcaccgtcttataaaccgaagaatctgctcagtagtaaacattgacatgccttccaggtattctccattgactccattttccttaaaagtttgccggtaactgccgaggtttatctcttccaaccacaaaccgacatcctcaacagtccaaatgaagaaatcaagaggctctggtgggtgttctttgctcatctccctcatcat

SEQ ID NO: 5 - DNA, 21 bp, Prunus persica
gattgtgaaggacacgtagct

SEQ ID NO: 6 - DNA, 23 bp, Prunus persica
tgtctgtaacttggctgtgttta

SEQ ID NO: 7 - DNA, 20 bp, Prunus persica
tgttgtttgggacatctgat

SEQ ID NO: 8 - DNA, 24 bp, Prunus persica
agcagattacatgaaaagtctcct

SEQ ID NO: 9 - DNA, 28 bp, Artificial Sequence
  chemical synthesized
atgtcgacggcgttatgatgagggagat

SEQ ID NO: 10 - DNA, 29 bp, Artificial Sequence
  chemical synthesized
atcccgggttatggttccagcttcaagga

SEQ ID NO: 11 - DNA, 412 bp, Artificial Sequence
  Chemically synthesized
atgtcgacggcgttatgatgagggagatgagcaaagaacacccaccagagcctcttgatttcttcatttggactgttgaggatgtcggtttgtggttggaagagataaacctcggcagttaccggcaaacttttaaggaaaatggagtcaatggagaatacctggaaggcatgtcaatgtttactactgagcagattcttcggtttataagacggtgccacatgaaatggggagactttatcacgctgtgtaaggaactaaggcgaattaaagtggcttgcctgaaaggggagcaaaaggtccgcaggccatggtgggctccatcttgcctctcagtagtatttgtcaaggtggcaaagcgtaacagacaatcacgggttgtttccttgaagctggaaccataacccgggat

SEQ ID NO: 12 - DNA, 24 bp, Artificial Sequence
  chemical synthesized
tgcctagagaacagagtaggaaag

SEQ ID NO: 13 - DNA, 25 bp, Artificial Sequence
  chemical synthesized
gaccagcgatagatacattaaaggc

SEQ ID NO: 14 - DNA, 23 bp, Artificial Sequence
  chemical synthesized
cgtgattgtctgttacgctttgc

SEQ ID NO: 15 - DNA, 24 bp, Artificial Sequence
  chemical synthesized
tcacgctgtgtaaggaactaaggc

SEQ ID NO: 16 - DNA, 20 bp, Prunus persica
aagaaatcaagaggctctgg

SEQ ID NO: 17 - DNA, 28 bp, Artificial Sequence
  synthesized
attgaagaaatcaagaggctctgggttt

SEQ ID NO: 18 - DNA, 24 bp, Artificial Sequence
  chemical synthesized
attgaagaaatcaagaggctctgg

SEQ ID NO: 19 - DNA, 24 bp, Artificial Sequence
  chemical synthesized
aaacccagagcctcttgatttctt

SEQ ID NO: 20 - protein, 129 aa, Malus domestica
MIREMSKEQPPEPLDFFIWTVEDVGLWLEEIKLGSYRQTFKENGVNGEYLEGMSMFTTEQILRFIRRCHMKWGDFITLCKELRRIKVACLKGEQKVRRPWWAPSCLSVVFVKVAKRNRQSRVVSLKLEP

SEQ ID NO: 21 - protein, 129 aa, Pyrus bretschneideri
MIRQMSKEQPPEPLDFFIWTVEDVGLWLEEIKLGSYRQTFKENGVNGEYLEGMSMFTTEQILRFIRRCHMKWGDFITLCKELRRIKVACLKGEQKVRRPWWAPSCLSVVFVKVAKRNRQSRVVSLKLEP

SEQ ID NO: 22 - protein, 129 aa, Prunus persica; identical to SEQ ID NO: 3

SEQ ID NO: 23 - protein, 125 aa, Citrus sinensis
MSKEKPPEPLDFFIWTVEDVGLWLEEINLGGYRQIFKENGVNGEYLEGMSMFTTEQILRFIRRCHMKWGDFITLCKELRRIKVACLKGEQKVRRPWWAPSCLSVVFVKVAKRNRQSRVVSLKLEP

SEQ ID NO: 24 - protein, 125 aa, Juglans regia
MSKEHPPEPLDFFIWTVEDVGLWLEEINLGSYRQIFKENGVNGEYLEGMSMFTTEQILRFIRRCHMKWGDFITLCKELRRIKVACLKGEQKVRRPWWAPSCLSVFFVKVAKHNRKSRVVSLKLEP

SEQ ID NO: 25 - protein, 125 aa, Populus_trichocarpa
MSKERPPEPLDFFIWTVEDVGLWLEEINLGSYRQIFKDNGVNGEYLEGMSMFTTEQILRFIRRCHMKWGDFITLCKELRRIKVACLKGEQKVRRPWWVPSCLSAIFVKVAKHNRQSRVVSLKLEP

SEQ ID NO: 26 - protein, 125 aa, Eucalyptus grandis
MTKEVPPEPLDFFIWTVEDVGLWLEEINLGGYRQVFKENGVNGEYLESMSMFTTEQILRFIRRCHMKWGDFITLCKELRRIKVACLKGEQKVRRPWWAPSCLSVFFVRVAKRNRQSRVVSLKLEP

SEQ ID NO: 27 - DNA, 378 bp, Malus domestica
atgagcaaagagcagccacctgagcctcttgatttcttcatttggactgttgaggatgttggtttgtggttggaagaaataaaactgggcagttaccggcaaacttttaaggaaaatggtgtcaatggagaatacctggaaggcatgtcaatgtttactactgagcagattcttcggtttataagacgttgccacatgaaatggggagactttatcacgctgtgtaaggaactaaggcgaattaaagtggcttgcctaaaaggggagcaaaaggtccgcagaccatggtgggctccgtcttgcctctcagtagtattcgtcaaggtggccaagcgtaacagacaatcacgagttgtttccttgaagctggaaccgtaa

SEQ ID NO: 28 - DNA, 378 bp, Pyrus bretschneideri; identical to SEQ ID NO: 27

SEQ ID NO: 29 - DNA, 378 bp, Prunus persica
atgagcaaagaacacccaccagagcctcttgatttcttcatttggactgttgaggatgtcggtttgtggttggaagagataaacctcggcagttaccggcaaacttttaaggaaaatggagtcaatggagaatacctggaaggcatgtcaatgtttactactgagcagattcttcggtttataagacggtgccacatgaaatggggagactttatcacgctgtgtaaggaactaaggcgaattaaagtggcttgcctgaaaggggagcaaaaggtccgcaggccatggtgggctccatcttgcctctcagtagtatttgtcaaggtggcaaagcgtaacagacaatcacgggttgtttccttgaagctggaaccataa

SEQ ID NO: 30 - DNA, 378 bp, Juglans regia
atgagcaaagaacacccacctgagcctctggatttcttcatttggactgttgaggatgttggtttgtggttggaagagataaatcttggtagttaccggcagatttttaaagaaaatggtgtcaacggagaatacctggaaggcatgtccatgtttactactgagcagattctacggtttataagacgatgccacatgaaatggggagacttcatcactctgtgcaaggagctcaggcggattaaagtggcttgcctgaaaggggaacaaaaggtccgccggccatggtgggctccatcttgcctctcggtattctttgtcaaggtggcaaaacacaacagaaagtcacgagttgtttccttgaagctggaaccgtga

SEQ ID NO: 31 - DNA, 378 bp, Populus_trichocarpa
atgagcaaagaaaggcctcctgagcctctcgatttcttcatttggactgttgaggatgttggtttgtggttagaagaaataaatcttggcagctaccgccaaatttttaaagataatggtgtgaacggagaatatctggaaggcatgtccatgttcacaactgaacagattttacggtttataaggcggtgccacatgaagtggggagacttcatcacactatgtaaggagctcagacgaataaaagtggcttgcctaaaaggagagcaaaaggttcgccggccatggtgggttccgtcctgcctctccgcaatctttgtcaaggttgcaaagcacaacagacagtcacgagttgtttccttgaagctggaaccatga

SEQ ID NO: 32 - DNA, 378 bp, Citrus sinensis
atgagcaaagagaagccacctgagccccttgatttctttatctggactgttgaggatgttggtttatggttggaagagataaatctgggtggctatcgtcagatcttcaaagagaatggtgtcaatggtgaatacttggagggcatgtcaatgttcacgactgaacagattcttcggtttataagacggtgccatatgaagtggggcgacttcattacattgtgtaaggaactcagacgaataaaagtggcttgtctaaaaggggagcaaaaagttcgccggccttggtgggctccgtcttgcctctctgtagtatttgtgaaggtggcaaagcgaaacagacagtcgcgagttgtttccttgaagctggaaccatga

SEQ ID NO: 33 - DNA, 378 bp, Eucalyptus grandis
atgaccaaagaagtgccgccggagcctctcgatttcttcatttggactgttgaggatgtaggtttgtggttggaagagataaatttgggtggctaccgccaagttttcaaggaaaatggtgtcaatggagaatatcttgagagcatgtctatgttcacgactgaacagatcctccggtttattaggcggtgccacatgaaatggggcgacttcatcacattatgtaaagagcttcgtcggattaaggtggcgtgtctaaaaggggagcagaaagtgcgccggccatggtgggctccttcttgcctttcagttttctttgtcagggtggcgaaacggaacaggcagtctcgggttgtttcactcaagctggaaccgtga

SEQ ID NO: 34 - DNA, 378 bp, Artificial Sequence
  consensus sequence
  misc_feature (93)..(93): n is a, c, g, or t
  misc_feature (111)..(111): n is a, c, g, or t
  misc_feature (219)..(219): n is a, c, g, or t
  misc_feature (315)..(315): n is a, c, g, or t
  misc_feature (336)..(336): n is a, c, g, or t
atgagcaaagaacagccacctgagcctcttgatttcttcatttggactgttgaggatgttggtttgtggttggaagagataaatctgggcagntaccggcaaatttttaangaaaatggtgtcaatggagaatacctggaaggcatgtcaatgtttactactgagcagattcttcggtttataagacggtgccacatgaaatggggagacttcatcacnctgtgtaaggaactcaggcgaattaaagtggcttgcctaaaaggggagcaaaaggtccgccggccatggtgggctccgtcttgcctctcagtagtntttgtcaaggtggcaaagcgnaacagacagtcacgagttgtttccttgaagctggaaccgtga

SEQ ID NO: 35 - protein, 125 aa, Artificial Sequence
  consensus sequence
  misc_feature (5)..(5): Xaa can be any naturally occurring amino acid
  misc_feature (35)..(35): Xaa can be any naturally occurring amino acid
MSKEXPPEPLDFFIWTVEDVGLWLEEINLGSYRQXFKENGVNGEYLEGMSMFTTEQILRFIRRCHMKWGDFITLCKELRRIKVACLKGEQKVRRPWWAPSCLSVVFVKVAKRNRQSRVVSLKLEP

SEQ ID NO: 36 - DNA, 1603 bp, Prunus persica
tctccccagtatcgctttgcaatccgatacaatatttatatggataaaaatgtaatttagtgttattatatgaaatttagctattggtcttaggaatcttaggattacctctgggaacagatattttgtgactaaaggggattattgatattaaaattaaacacagccaagttacagacaaaaacatttctttgccatgtgaagtaatgccgtttgatatggatgaactctagtcggataatctcaaaagaaagagtttaaattgccctaagtttaaggttgtcctgaacaaaaatttgggtgaatattgttttttttatttttatttttgggttggagggactgttatattataacagtgacttaataatagcctgagtttatcatgtgccctgatgaaggcctcatgtacatattttttggccatatataataaaacgaatataaagatacatggctcacccacatgtattctcatgtttattttcccctttcgtacaggatgtcggtttgtggttggaagagataaacctcggcagttaccggcaaacttttaaggaaaatggagtcaatggagaatacctggaaggcatgtcaatgtttactactgagcagattcttcggtttataagacggtgccacatgaaatggggagactttatcacgctgtgtaaggaactaaggcgaattaaaggtctgtagcctagaaattttcctagcagtctttatcatgctgtgaaaaaaatgtctttgtgttcgttcttatatcaaaaatttcatttagtctctggagttattggataaatagatttgaataagggttgtgattatcttactgttgcattttctatgcacaggtacatatgacatttgattattggaaaatataattattccaaaagtaaaacagaaaagagagtttttgtagagtatatggattggtgagatagactggccatgcatgatccattcatttcatgcactcaaatgtctactgatagcatagcacgctgacactaagaaaccgttcttatgcctcattcaatagcattgtgaaactctctcttgatctcattatctatcctagcaagctgagaatttaaacctttctggaatcatatttcccttgagctacatatatatgcctttttcctctgaaatttgaattgcatttagataccatctgtgtgtgtgctaaaaccaagtttatgtccagtggcttgcctgaaaggggagcaaaaggtccgcaggccatggtgggctccatcttgcctctcagtagtatttgtcaaggtggcaaagcgtaacagacaatcacgggttgtttccttgaagctggaaccataaaggatgatcttatatgtacctacatatattttctttatttctttttcttttttttgcctagagaacagagtaggaaagtacttttttcctttcttttttttctctcgagtctgtcaaattacatgttcgttcttctttggagaactctgtttttatatagcctttaatgtatctatcgctggtcttttgatctttatatgtttgtttactttgagaaccatgatgaggcaatcaagttcttgttgcagatcatcca

SEQ ID NO: 37 - DNA, 20 bp, Malus domestica
aagaaatcaagaggctcagg

SEQ ID NO: 38 - DNA, 20 bp, Populus_trichocarpa
aagaaatcgagaggctcagg

SEQ ID NO: 39 - DNA, 20 bp, Juglans nigra
aagaaatccagaggctcagg

SEQ ID NO: 40 - DNA, 20 bp, Citrus sinensis
aagaaatcaaggggctcagg

SEQ ID NO: 41 - DNA, 20 bp, Eucalyptus grandis
aagaaatcgagaggctccgg

(19) United States
(12) Patent Application Publication
(10) Pub. No.: US 2019/0062381 A1
(43) Pub. Date: Feb. 28, 2019

(54) GENETICALLY ALTERED PLANTS HAVING WEEPING PHENOTYPE

(71) Applicant: The United States of America, as represented by the Secretary of Agriculture, Washington, DC (US)

(72) Inventors: Christopher D. Dardick, Shenandoah Junction, WV (US); Ralph Scorza, Shepherdstown, WV (US); Courtney A. Hollender, East Lansing, MI (US)

(21) Appl. No.: 16/103,103

(22) Filed: Aug. 14, 2018

Related U.S. Application Data
(60) Provisional application No. 62/546,062, filed on Aug. 16, 2017.

(51) Int. Cl. C07K 14/415; C12N 15/113; C12N 15/82; C12N 15/62

(52) U.S. Cl. CPC C07K 14/415; C12N 15/113; C12N 15/62; C12N 15/8201; C12N 15/8218

(57) ABSTRACT

Genetically altered eudicots that have the altered phenotype of weeping are provided. The genetically altered eudicots contain a genetic alteration that silences the expression of the WEEP gene or that results in production of non-functional WEEP protein or that results in production of a reduced amount of functional WEEP protein compared to the amount of functional WEEP protein produced by a wild-type eudicot with a non-weeping phenotype. Methods of producing such genetically altered eudicots are provided.

### CROSS REFERENCE TO RELATED APPLICATIONS

This patent application claims priority to U.S. patent application Ser. No. 62/546,062 filed on Aug. 16, 2017, contents of which are expressly incorporated by reference herein.

### SEQUENCE LISTING

The Sequence Listing submitted via EFS-Web as ASCII compliant text file format (.txt) filed on Aug. 14, 2018, named “SequenceListing_ST25”, (created on Aug. 10, 2018, 27 KB), is incorporated herein by reference. This Sequence Listing serves as paper copy of the Sequence Listing required by 37 C.F.R. § 1.821(c) and the Sequence Listing in computer-readable form (CRF) required by 37 C.F.R. § 1.821(e). A statement under 37 C.F.R. § 1.821(f) is not necessary.

### BACKGROUND OF THE INVENTION

### Field of the Invention

This invention relates to a gene Ppa013325, WEEP, identified from peach and its role in gravitropic sensing and shoot orientation in various eudicots. This invention also relates to methods of generating the weeping phenotype through RNAi-mediated silencing of Ppa013325 and/or null mutation of the gene and/or the generation of non-functional WEEP protein.

### Description of the Relevant Art

Weeping growth habits in both angiosperm and gymnosperm species have long been enjoyed as horticultural objects of beauty. Weeping phenotypes, which can vary in appearance from drastic drooping to mild spreading, have generally been attributed to either a lack of branch structural integrity or, in some instances, defects in gravitropism resulting in rootward branch growth. It has been proposed that weeping, or pendulous, shoot architectures may allow for novel fruit tree training methods, although to-date few weeping fruit trees have been commercialized. See, Bassi, et al., 1994. J. Am. Soc. Hortic. Sci. 119 (3):378-382; Werner and Chaparro, 2005. HortScience 40 (1):18-20; Chaparro, et al., 1994. TAG Theor. Appl. Genet 87 (7):805-815; and Bassi and Rizzo, 2000. Acta Hortic., 538 (1):411-414.

A weeping peach tree (Prunus persica) with normal flower and fruit development has been described (Monet, et al., 1988. Agronomie 8:127-132; Bassi, et al., 1994. J. Am. Soc. Hort. Sci. 119:378-382). Their shoots initially grow upwards, away from the gravity vector and then, for an as-of-yet unknown reason, they arch and grow downwards. Interestingly, weeping peach branches do not have as obvious a lack of rigidity in contrast to the drooping whip-like branches of weeping willow trees (Salix babylonica). After the downward shoot growth is initiated in a weeping peach shoot, subtending buds are released from dormancy and will subsequently grow in the same arching manner in a cascading pattern.

Previously, the architecture of weeping peach and cherry trees (Prunus spachiana), which exhibits a similar phenotype, was linked to abnormalities associated with the growth hormone gibberellic acid (GA), as well as to reduced mechanical rigidity resulting from a disruption of tension wood formation (Reches, et al., 1974. New Phytol. 73:841-846; Nakamura, et al., 1994. Plant Cell Physiol. 35 (3):523-527; Baba, et al., 1995. Plant Cell Physiol. 36:983-988; Nakamura, et al., 1995. Acta Hortic. 394:272-280; Sugano, et al., 2004. Seibutsu Kagaku 18:261-266). Aside from hormone-related investigations, an understanding of the biology behind weeping tree phenotypes is minimal. Genetic studies, however, have been performed with some weeping trees, but no causative alleles have been identified. The eastern redbud has two recessive non-allelic weeping varieties: Covey (Cercis canadensis L.), which resembles the weeping peach growth habit, and the spreading variety Traveller (Cercis Canadensis var. texensis (Roberts, et al., 2015. Hortic. Res. 2:15049). Additionally five weeping chestnut varieties have also been linked to a single recessive locus while a sixth is controlled by a single dominant allele (Kotobuki, et al., 2005. Proc. III^{rd} Intl. Chestnut Congr., Acta Hort. 693:477-484). Weeping apple phenotypes have also been linked to a single dominant allele (Sampson and Cameron, 1965. Proc. Am. Soc. Hortic. Sci. 86:717-722; Tsuchiya and Soejima, 1986. Japan. Soc. Hort. Sci. Autumn Meet., 112-113). A weeping tree allele was localized in the Japanese apricot (Prunus mume) weeping to a region on linkage group 7 that contains 159 genes including 69 candidates based on amino acid polymorphisms (Zhang, et al., 2015. Nat. Commun. 3:1318).

The peach weeping phenotype has been associated with a recessive locus named pl (for pleurer, the French word for weeping) (Monet, et al., supra; Bassi, et al., supra; Chaparro, et al., 1994. TAG Theor. Appl. Genet. 87:805-815; Bassi and Rizzo, supra). Using RAPD markers, pl was placed on linkage group two of an early peach genetic map (Dirlewanger and Bodo, 1994. Euphytica 77:101-103); however, the markers used were not incorporated into the peach genome (Dirlewanger and Bodo, 1994. Euphytica 77 (1-2):101-103). Thus, both the identification and the location of pl remains unknown. See, Verde, et al., 2013. Nat. Publ. Gr. 45:487-494.

### SUMMARY OF THE INVENTION

The causative nucleic acid molecule for a weeping phenotype in peach and plum has been identified as Ppa013325 cDNA (SEQ ID NO: 2) and confirmed that silencing its expression via a loss of function mutation results in the creation of the weeping appearance in Prunus tree species. The gene is called WEEP, and the protein encoded therein is WEEP (SEQ ID NO: 3). Eudicots contain a genomic WEEP, and the cDNA encoding WEEP and WEEP itself have 95% or greater identity to SEQ ID NO: 34 (DNA consensus sequence) and SEQ ID NO: 35 (amino acid consensus sequence), respectively.

It is an object of the invention to have a dsRNA containing at least any contiguous 19 nucleotides of WEEP, a linker, and a sequence complementary to the at least any contiguous 19 nucleotides of WEEP. It is another object of this invention that WEEP encode the consensus WEEP having amino acid sequence of SEQ ID NO: 35, and/or that WEEP has a consensus sequence of SEQ ID NO: 34. It is another object that WEEP has the amino acid sequence of SEQ ID NO: 20, 21, 22, 23, 24, 25, or 26; or that WEEP has the DNA sequence of SEQ ID NO: 27, 28, 29, 30, 31, 32, or 33. It is a further object of this invention that the dsRNA contains SEQ ID NO: 4 (an antisense sequence) and its complementary sequence (sense sequence). It is a further object of this invention to have an expression vector that contains a heterologous promoter operably linked to a polynucleotide encoding the dsRNA. It is another object of this invention to have a transformed plant cell that contains the expression vector and produces the dsRNA.

It is an object of this invention to have a genetically altered eudicot plant and progeny thereof which have a weeping phenotype compared to the non-weeping phenotype of a wild-type eudicot plant. It is another object of this invention that the genetically altered eudicot plant contains a genetic alteration that reduces the amount of functional WEEP protein produced by the genetically altered plant compared to amount of functional WEEP protein produced by the wild-type eudicot plant and that the reduced amount of functional WEEP protein causes the genetically altered eudicot plant and progeny thereof to have the weeping phenotype. It is an object of this invention that the genetic alteration can be (i) a null mutation in WEEP; (ii) a deletion of WEEP from the plant's genome (but this deletion mutation is not SEQ ID NO: 36); and/or (iii) an expression vector that produces the dsRNA discussed supra. It is another object of this invention that the null mutation in WEEP can be a stop codon replacing a codon encoding an amino acid or other alteration in WEEPs coding sequence so that a non-functional WEEP is produced. One potential alteration is changing or deleting the ATG codon at nucleotides 1-3 of SEQ ID NO: 2. It is another object of this invention to have a pollen, leaf, stem, flower, seed, cell, and/or germplasm of the genetically altered eudicot. The eudicot can be a wood shrub or tree. The tree can be Malus spp., Pyrus spp., Prunus spp., Juglans spp., Populus spp., Citrus spp., Eucalyptus spp., or any other type of fruit bearing or non-fruit bearing tree.

It is another object of this invention to generate a genetically altered eudicot plant having a weeping phenotype compared to the wild-type eudicot plant having a non-weeping phenotype where the genetic alteration causes the weeping phenotype because the genetic alteration causes a reduced amount of functional WEEP to be produce by the genetically altered eudicot plant compared to the amount of functional WEEP produced by the wild-type eudicot plant. It is another object of this invention that the genetic alteration can be made by the steps of (i) creating a genetic alteration in a wild-type eudicot plant cell's genome to generate a transformed eudicot cell, (ii) selecting at least one transformed eudicot cell that expresses the genetic alteration to produce at least one selected genetically altered eudicot cell, and (iii) inducing the selected genetically altered eudicot cell to grow into a genetically altered eudicot plant that expresses the genetic alteration. It is another object of this invention that genetic alteration in the wild-type eudicot cell's genome is made by transforming the wild-type eudicot plant cell with an expression vector that contains a heterologous promoter operably linked to a polynucleotide encoding a dsRNA, and the polynucleotide contains any contiguous 19 nucleotides of WEEP, a linker, and a sequence complementary to the at least any contiguous 19 nucleotides of WEEP. This dsRNA and the expression vector encoding it is described supra.

It is another object of this invention that the method of creating the genetic alteration (described supra) is made by inducing a targeted cleavage event in the wild-type eudicot plant cell to generate a genetically altered WEEP which causes production of an altered WEEP having reduced functionality compared to wild-type WEEP's functionality and, thus, causes the weeping phenotype. It is another object of this invention that one can induce the targeted cleavage event by transforming the wild-type plant eudicot cell with an expression vector encoding a RNA-guided DNA endonuclease (such as Cas9) and a polynucleotide encoding a sgRNA that causes the genetic alteration in WEEP. It is a further object of this invention that WEEP encodes the consensus WEEP having amino acid sequence of SEQ ID NO: 35, and/or that WEEP has a consensus sequence of SEQ ID NO: 34. It is another object of this invention that WEEP has the amino acid sequence of SEQ ID NO: 20, 21, 22, 23, 24, 25, or 26; or that WEEP has the DNA sequence of SEQ ID NO: 27, 28, 29, 30, 31, 32, or 33. It is a further object of this invention that the sgRNA has a sequence of approximately 20 nucleotides of WEEP that encodes WEEP with consensus sequence of SEQ ID NO: 35. It is a further object of this invention that the sgRNA has a sequence of approximately 20 nucleotides of WEEP—consensus sequence SEQ ID NO: 34, or any of SEQ ID NO: 27, 28, 29, 30, 31, 32, or 33. It is another object of this invention that sgRNA has the sequence of SEQ ID NOs: 16, 37, 38, 39, 40, or 41.

It is an object of this invention to have a genetically altered eudicot with a weeping phenotype that is produced by the methods described supra, and such that the amount of functional WEEP produced by the genetically altered eudicot is less than the amount of functional WEEP produced by wild-type eudicot that does not have the weeping phenotype. It is a further object of this invention to have a pollen, leaf, stem, flower, seed, cell, and/or germplasm of the genetically altered eudicot made by these methods. The eudicot can be a wood shrub or tree. The tree can be Malus spp., Pyrus spp., Prunus spp., Juglans spp., Populus spp., Citrus spp., Eucalyptus spp., or any other type of fruit bearing or non-fruit bearing tree.

### BRIEF DESCRIPTION OF THE DRAWINGS

FIG. 1A shows the p-nome map of DNA variants and corresponding map position on chromosome #3 for the allele of peach gene Ppa013325. Dots represent single variants. Broad peak indicated with the bracket shows initial mapped region.

FIG. 1B shows the genomic sequence spanning from 15,604,111 to 15,601,132 (SEQ ID NO: 1). The italicized genomic sequences are absent in the naturally occurring weeping trees. The ATG start codon and TAA stop codon are underlined.

FIG. 2 shows maximum likelihood phylogenetic tree of WEEP proteins.

FIG. 3A shows WEEP protein alignment for the indicated eudicot trees and contains protein ID numbers. FIG. 3B, 3C, and 3D show WEEP DNA alignment for indicated eudicot trees and contains gene ID numbers.

FIG. 4A shows the relative WEEP expression levels for the indicated transformed plum lines (RNAi silencing of WEEP) and negative control. Bars represent biological replicate standard deviations. FIG. 4B shows transformed plum trees (lines 1, 5, 6, 9, and 10) containing a WEEP silencing vector at the end of their 1^{st} growing season. VC is negative control plant. FIG. 4C shows transformed plum trees (lines 1, 5, 6, and 10) containing a WEEP silencing vector at the end of their 2^{nd} growing season.

FIG. 5A shows relative expression of WEEP in dissected tissues from ˜2 year old standard peach trees grown in pots in a greenhouse. Expression values determined by qPCR based on a total RNA standard curve. Error bars represent standard deviation from two and four biological replicates for each tissue. Biological replicate values are from three technical replicates. FIG. 5B shows the relative expression of WEEP in dissected shoot tissues taken from standard peach trees in the field grown mapping population. Expression values determined by qPCR based on a total RNA standard curve. Error bars represent standard deviation from two and four biological replicates for each tissue. Biological replicate values are from three technical replicates.

### DETAILED DESCRIPTION OF THE INVENTION

Naturally occurring weeping traits have been bred into numerous tree species largely for ornamental purposes. These weeping tree forms are extremely popular for use in landscape design. However, in the absence of naturally occurring traits, weeping forms are simply unavailable for many tree species and woody shrubs. In such cases, a weeping phenotype would be highly desirable. The invention described here would enable the creation of weeping phenotype for a wide range of tree species and woody shrubs.

Herein the following are described. (1) A mutated gene (WEEP) in peach that causes the weeping phenotype. (2) The use of RNAi silencing in genetically modified plants to silence the expression of the naturally occurring WEEP gene, thereby causing the weeping phenotype. (3) The use of CRISPR/Cas9 to generate a genetically altered plant that has a null mutation in WEEP whereby the genetically altered plant does not produce a functional WEEP protein and has the weeping phenotype. (4) Evidence that WEEP is a highly conserved ancient gene.

This invention involves a novel and unexpected method of generating genetically altered trees and/or wood shrubs that have a weeping phenotype (compared to the non-weeping phenotype of wild-type trees and/or woody shrubs) by manipulating the expression and/or translation of WEEP via RNAi and/or reducing the amount of functional WEEP protein present in the genetically altered tree or woody shrub. By altering the expression and/or translation of WEEP and/or reducing the amount of functional WEEP, one causes the genetically altered tree or wood shrub to have the weeping phenotype. For the purposes of this invention, the terms “function”, “functional”, and “functionality” include any activity that the protein or other compound possesses. A protein may have enzymatic activity, binding activity, transporting activity, structural activity, etc. The italicized “WEEP” refers to the gene; the non-italicized “WEEP” refers to the protein encoded by the WEEP gene.

As mentioned above, one embodiment of this invention involves using RNAi to reduce production of WEEP protein which causes a weeping phenotype in the genetically altered plant (tree and/or wood shrub). In another embodiment, the invention involves altering the genomic WEEP sequence such that the encoded protein lacks functionality or has reduced functionality compared to the activity of non-modified WEEP protein activity. Of course, such genetically altered plant possessing WEEP protein with reduced or no functionality are another embodiment of this invention.

Plant shoots typically grow upwards—against the gravity vector and towards light. However, the naturally occurring weeping peach growth phenotype, with arched branches and downward-growing shoots, contradicts this phenomenon. The underlying reason for this abnormal gravitropic growth habit is poorly understood. The identification of an allele of Ppa013325 as the causative allele for the weeping peach phenotype sheds light on this subject.

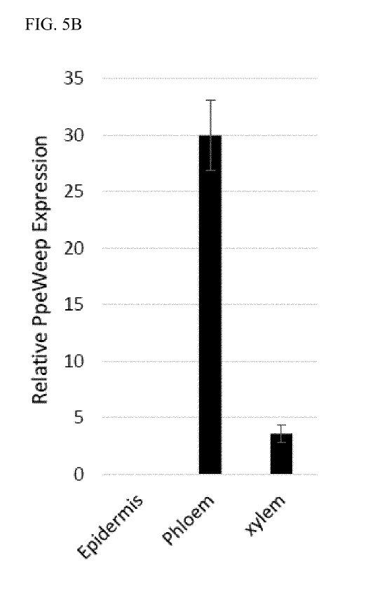

In peach, WEEP was most prominently expressed in hand-dissected shoot vascular tissues (FIGS. 5A and 5B). This localization strengthens the hypothesis that WEEP is needed for gravity sensing, signaling, or response. Gravity sensing in shoots occurs in the endodermis (Fukaki et al. 1998. Plant J. 14:425-430; Hashiguchi et al. 2013. Am. J. Bot. 100:91-100). Endodermal cells have highly lignified cell walls and contain starch-filled amyloplasts that function as statoliths (Hashiguchi, et al., supra; Fukaki, et al., supra; Masson, et al., 2002. Arab. B. 1:e0043; Tasaka, et al., 1999. Trends Plant Sci. 4 (3):103-107; Gerttula, et al., 2015. Plant Cell 27 (10):2800-2813; and Groover, A., 2016. New Phytol. 211:790-802). Numerous studies have shown that plants lacking endodermal tissue, such as the short root (shr)/shoot gravitropism 1 (sgrl) and scarecrow (scr)/shoot gravitropism 7 (sgr7) mutants, lack or have impaired and reduced gravitropic responses in their shoots (Hashiguchi, et al., supra; and Masson, et al., supra).

Because this invention involves biotechnology, the following definitions are provided to assist in understanding this invention.

The terms “isolated”, “purified”, or “biologically pure” as used herein, refer to material that is substantially or essentially free from components that normally accompany the material in its native state or when the material is produced. In an exemplary embodiment, purity and homogeneity are determined using analytical chemistry techniques such as polyacrylamide gel electrophoresis or high performance liquid chromatography. A nucleic acid or particular bacteria that are the predominant species present in a preparation is substantially purified. In an exemplary embodiment, the term “purified” denotes that a nucleic acid or protein that gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

As used herein, the terms “nucleic acid molecule”, “nucleic acid sequence”, “polynucleotide”, “polynucleotide sequence”, “nucleic acid fragment”, “isolated nucleic acid fragment” are used interchangeably herein. These terms encompass nucleotide sequences and the like. DNA and RNA are nucleic acids.

The term “isolated” polynucleotide refers to a polynucleotide that is substantially free from other nucleic acid sequences, such as other chromosomal and extrachromosomal DNA and RNA, that normally accompany or interact with it as found in its naturally occurring environment. However, isolated polynucleotides may contain polynucleotide sequences which may have originally existed as extrachromosomal DNA but exist as a nucleotide insertion within the isolated polynucleotide. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

Unless otherwise indicated, a particular nucleic acid sequence for each amino acid substitution (alteration) also implicitly encompasses conservatively modified variants thereof (e.g., degenerate codon substitutions), the complementary (or complement) sequence, and the reverse complement sequence, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)). Because of the degeneracy of nucleic acid codons, one can use various different polynucleotides to encode identical polypeptides. Table 1, infra, contains information about which nucleic acid codons encode which amino acids and is useful for determining the possible nucleotide substitutions that are included in this invention.

TABLE 1

| Amino acid | Nucleic acid codons | Amino acid | Nucleic acid codons |
|---|---|---|---|
| Ala/A | GCT, GCC, GCA, GCG | Leu/L | TTA, TTG, CTT, CTC, CTA, CTG |
| Arg/R | CGT, CGC, CGA, CGG, | Lys/K | AAA, AAG |
| | AGA, AGG | | |
| Asn/N | AAT, AAC | Met/M | ATG |
| Asp/D | GAT, GAC | Phe/F | TTT, TTC |
| Cys/C | TGT, TGC | Pro/P | CCT, CCC, CCA, CCG |
| Gln/Q | CAA, CAG | Ser/S | TCT, TCC, TCA, TCG, AGT, AGC |
| Glu/E | GAA, GAG | Thr/T | ACT, ACC, ACA, ACG |
| Gly/G | GGT, GGC, GGA, GGG | Trp/W | TGG |
| His/H | CAT, CAC | Tyr/Y | TAT, TAC |
| Ile/I | ATT, ATC, ATA | Val/V | GTT, GTC, GTA, GTG |
| Stop | TAA, TGA, TAG | | |

The term “primer” refers to an oligonucleotide, which is capable of acting as a point of initiation of synthesis when placed under conditions in which primer extension is initiated. A primer may occur naturally, as in a purified restriction digest, or may be produced synthetically.

A primer is selected to be “substantially complementary” to a strand of specific sequence of the template. A primer must be sufficiently complementary to hybridize with a template strand for primer elongation to occur. A primer sequence need not reflect the exact sequence of the template. For example, a non-complementary nucleotide fragment may be attached to the 5′ end of the primer, with the remainder of the primer sequence being substantially complementary to the strand. Non-complementary bases or longer sequences can be interspersed into the primer, provided that the primer sequence is sufficiently complementary with the sequence of the template to hybridize and thereby form a template primer complex for synthesis of the extension product of the primer.

“dsRNA” refers to double-stranded RNA that comprises a sense region and an antisense region of a selected target gene (or sequences with high sequence identity thereto so that gene silencing can occur), as well as any smaller double-stranded RNAs formed therefrom by RNAse or Dicer activity. Such dsRNA can include portions of single-stranded RNA, but contains at least 18 base pairs of dsRNA. A dsRNA after been processed by Dicer generates siRNAs (18-25 bp in length) that are double-strand, and could contain ends with 2 nucleotide overhangs, which will be single-stranded. It is predicted that usually siRNA around 21 nt in length (or, alternatively, between 17 and 27 nt in length), will be incorporated into RISC. In one embodiment, the sense region and the antisense region of a dsRNA are on the same strand of RNA and are separated by a linker. In this embodiment, when the sense region and the antisense region anneal together, the dsRNA contains a loop which is the linker. One promoter operably linked to the DNA or RNA encoding both the sense region and the antisense region is used to produce the one RNA molecule containing both the sense region and the anti-sense region. In another embodiment, the sense region and the antisense region are present on two distinct strands of RNA (a sense strand and the anti-sense strand which is complementary to the sense strand) which anneal together to form the dsRNA. In this embodiment, a promoter is operably linked to each strand of DNA or RNA; where one DNA or RNA strand encodes the RNA containing the sense region and the other strand of DNA or RNA encodes the RNA containing the anti-sense region. In this embodiment, the promoter on each strand can be the same as or different from the promoter on the other strand. After the RNAs are transcribed, two RNA strands anneal together because the sense region and the anti-sense region are complementary to each other, thus forming the dsRNA. In yet another embodiment, one strand of DNA or RNA can encode both the sense region and the anti-sense region of the dsRNA. However, the DNA or RNA coding each region are separated from each other so that two promoters are necessary to transcribe each region. That is, the DNA or RNA encoding the anti-sense region and the DNA or RNA encoding the sense region are operably linked to their own promoter. Again, the two promoters can be the same promoter or different promoters. In one embodiment, the promoter can be a T7 RNA polymerase promoter. Other promoters are well-known in the art and can be used (see discussion infra). While many embodiments of this invention use DNA to encode the sense region and/or anti-sense region, as described infra, it is possible to use a recombinant RNA virus to produce the dsRNA described herein. In such cases, a virus has had its genome altered so that the infected cell produces WEEP sequence described herein or the reverse complement thereof or both.

Active dsRNA molecules have worked when they were as long as 1,000 bp, and should work when even longer. For the purposes of the inventions described herein, any siRNA having at least 19 nt length derived from SEQ ID NO: 2 or the reverse complement sequence of SEQ ID NO: 2 will be specific to WEEP. In one embodiment, the dsRNA can be any 19, 20, 21, 22, 23, 24, 25, 26, 27, 28, 29, 30, 31, 32, 33, 34, 35, 36, 37, 38, 39, 40, 45, 50, 55, 60, or longer contiguous nucleotides, up and including the full-length of WEEP cds (SEQ ID NO: 2). In another embodiment, the reverse complement sequence of WEEP can be SEQ ID NO: 4. In alternative embodiments, the dsRNA can range in length between 19 bp and 30 bp, between 19 bp and 28 bp, and between 21 bp and 28 bp. In yet another embodiment, RNA forms that are created by RNAse III family (Dicer or Dicer-like ribonuclease) or Dicer activity that are longer dsRNA are within the scope of this invention.

One can use computer programs to predict dsRNA sequences that will be effective in reducing production of the desired gene/protein (in this embodiment WEEP). Information about such computer programs can be found on the websites for the following entities: Gene Link (genelink.com/siRNA/RNAiwhatis.asp); and The RNAi Web (rnaiweb.com/RNAi/RNAi_Web_Resources/RNAi_Tools Software/Online_siRNA Design_To ols/index.html). Using such computer programs, one can obtain sequences that differ from SEQ ID NO: 2 which can be used to generate dsRNA via binding to WEEP mRNA. Alternatively, one can determine an appropriate sequence to test using the methodologies described in Preuss, S. and Pikaard, C. S. (2003) Targeted gene silencing in plants using RNA interference, in RNA interference (RNAi)˜Nuts and Bolts of siRNA Technology (Engelke, D., Ed.), pp 23-36, DNA Press, LLC.

siRNA can be synthetically made, expressed and secreted directly from a transformed cell, or microbe, or can be generated from a longer dsRNA by enzymatic activity. These siRNAs can be blunt-ended or can have 1 bp to 4 bp overlapping ends of various nucleotide combinations. Also modified microRNAs comprising a portion of WEEP and its reverse complementary sequence are included herein as dsRNAs. In one embodiment of the invention, the dsRNA is expressed in a plant to be protected, or expressed in microorganisms which can be endemic organisms of the plant (microbes, virus, phytoplasma, viroids, fungal, protists) or free-living microbes (yeasts, bacteria, protists, fungi) any of which are delivered, alive, dead or processed, via root treatments, or foliar sprayed on plants, or injected into plants, which are to be protected. Alternatively, the microorganism can be a transgenic organism endemic to the plant and deliver dsRNA to the plant. See, e.g., Subhas, et al. (2014) J. Biotech. 176:42-49 for an example of virus induced gene silencing using Citrus tristeza virus. See, also, Tenllado, et al. (2003) BMC Biotechnol 3:3 for an example of a crude extract of a bacterial cell culture containing dsRNA that protects plants against viral infections.

In one embodiment, a dsRNA solution is administered to a woody shrub or tree. A dsRNA solution contains one or more of the dsRNAs discussed herein and an agriculturally acceptable carrier. An agriculturally acceptable carrier can be water, one or more liposomes, one or more lipids, one or more surfactants, one or more proteins, one or more peptides, one or more nanotubes, chitin, and/or one or more inactivated microorganisms that encapsulate the dsRNA. See WO 2003/004644 for examples of other agriculturally acceptable carriers. The dsRNA solution can also contain one or more sugars, compounds that assist in preventing dsRNA degradation, translaminar chemicals, chemical brighteners, clays, minerals, and/or fertilizers. One can spray the dsRNA solution on plants (leaves, branches, trunk, exposed roots, etc.). One can apply the dsRNA solution to the soil around the plant so that the plant's roots absorb the dsRNA solution and transport it to other parts of the plants. Alternatively, one or more roots can be placed in a container which contains the dsRNA solution so that those roots absorb the dsRNA solution. The dsRNA solution can also be injected into the plant. As such, the dsRNA solution can be in a spray dsRNA solution, a drenching dsRNA solution, or an injectable dsRNA solution. The dsRNA can also be mixed into irrigation water which is then administered to the plants. The plant's roots will absorb the dsRNA in the irrigation water, resulting in RNAi. Other types of solutions are known in the art.

The “complement” of a particular polynucleotide sequence is that nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff's rules (A<>T; G<>C) and reading in the 5′ to 3′ direction, i.e., in opposite direction of the represented nucleotide sequence (reverse complement).

In one embodiment of the invention, sense and antisense RNAs and dsRNA can be separately expressed in-vitro or in-vivo. In-vivo production of sense and antisense RNAs can use different chimeric polynucleotide constructs using the same or different promoters or using an expression vector containing two convergent promoters in opposite orientation. These sense and antisense RNAs which are formed, e.g., in the same host cells, or synthesized, and can then combine to form dsRNA. It is clear that whenever reference is made herein to a dsRNA chimeric or fusion polynucleotide or a dsRNA molecule, that such dsRNA formed, e.g., in plant cells, from sense and antisense RNA produced separately is also included. Also synthetically made dsRNA annealing RNA strands are included herein when the sense and antisense strands are present together.

As used herein, the term “promoter” refers to a polynucleotide that, in its native state, is located upstream or 5′ to a translational start codon of an open reading frame (or protein-coding region) and that is involved in recognition and binding of RNA polymerase and other proteins (trans-acting transcription factors) to initiate transcription. A “plant promoter” is a native or non-native promoter that is functional in plant cells, even if the promoter is present in a microorganism that infects plants or a microorganism that does not infect plants. The promoters that are predominately functional in a specific tissue or set of tissues are considered “tissue-specific promoters”. A plant promoter can be used as a 5′ regulatory element for modulating expression of a particular desired polynucleotide (heterologous polynucleotide) operably linked thereto. When operably linked to a transcribable polynucleotide, a promoter typically causes the transcribable polynucleotide to be transcribed in a manner that is similar to that of which the promoter is normally associated.

Plant promoters can include promoters produced through the manipulation of known promoters to produce artificial, chimeric, or hybrid promoters. Such promoters can also combine cis-elements from one or more promoters, for example, by adding a heterologous regulatory element to an active promoter with its own partial or complete regulatory elements. The term “cis-element” refers to a cis-acting transcriptional regulatory element that confers an aspect of the overall control of gene expression. A cis-element may function to bind transcription factors, trans-acting protein factors that regulate transcription. Some cis-elements bind more than one transcription factor, and transcription factors may interact with different affinities with more than one cis-element.

The term “recombinant” when used with reference, e.g., to a cell, or nucleic acid, protein, or vector, indicates that the cell, organism, nucleic acid, protein or vector, has been modified by the introduction of a heterologous nucleic acid or protein or the alteration of a native nucleic acid or protein, or that the cell is derived from a cell so modified. Thus, for example, recombinant cells may express genes/polynucleotides that are not found within the native (non-recombinant or wild-type) form of the cell or express native genes in an otherwise abnormal amount—over-expressed, under-expressed or not expressed at all—compared to the non-recombinant or wild-type cell or organism. In particular, one can alter the genomic DNA of a wild-type plant by molecular biology techniques that are well-known to one of ordinary skill in the art and generate a recombinant plant.

The term “vector” refers to DNA, RNA, a protein, or polypeptide that are to be introduced into a host cell or organism. The polynucleotides, protein, and polypeptide which are to be introduced into a host can be therapeutic or prophylactic in nature; can encode or be an antigen; can be regulatory in nature; etc. There are various types of vectors including viruses, viroids, plasmids, bacteriophages, cosmids, and bacteria.

An expression vector is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s).

A heterologous polynucleotide sequence is operably linked to one or more transcription regulatory elements (e.g., promoter, terminator and, optionally, enhancer) such that the transcription regulatory elements control and regulate the transcription and/or translation of that heterologous polynucleotide sequence. A cassette can have the heterologous polynucleotide operably linked to one or more transcription regulatory elements. As used herein, the term “operably linked” refers to a first polynucleotide, such as a promoter, connected with a second transcribable polynucleotide, such as a gene of interest, where the polynucleotides are arranged such that the first polynucleotide affects the transcription of the second polynucleotide. In some embodiments, the two polynucleotide molecules are part of a single contiguous polynucleotide. In other embodiments, the two polynucleotides are adjacent. For example, a promoter is operably linked to a gene of interest if the promoter regulates or mediates transcription of the gene of interest in a cell. Similarly a terminator is operably linked to the polynucleotide of interest if the terminator regulates or mediates transcription of the polynucleotide of interest, and in particular, the termination of transcription. Constructs of the present invention would typically contain a promoter operably linked to a transcribable polynucleotide operably linked to a terminator.

The terms “transgenic”, “transformed”, “transformation”, and “transfection” are similar in meaning to “recombinant”. “Transformation”, “transgenic”, and “transfection” refer to the transfer of a polynucleotide into a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur the organism or cell may need to be kept under certain conditions. Genetically altered organisms or cells containing the recombinant polynucleotide can be referred to as “transgenic” or “transformed” organisms or cells or simply as “transformants”, as well as recombinant organisms or cells.

A genetically altered organism is any organism with any changes to its genetic material, whether in the nucleus or cytoplasm (organelle). As such, a genetically altered organism can be a recombinant or transformed organism. A genetically altered organism can also be an organism that was subjected to one or more mutagens or the progeny of an organism that was subjected to one or more mutagens and has mutations in its DNA caused by the one or more mutagens, as compared to the wild-type organism (i.e., organism not subjected to the mutagens). Also, an organism that has been bred to incorporate a mutation into its genetic material is a genetically altered organism.

Transformation and generation of genetically altered monocotyledonous and dicotyledonous plant cells is well known in the art. See, e.g., Weising, et al., Ann. Rev. Genet. 22:421-477 (1988); U.S. Pat. No. 5,679,558; Agrobacterium Protocols, ed: Gartland, Humana Press Inc. (1995); and Wang, et al. Acta Hort. 461:401-408 (1998).

Examples of methods of plant transformation include Agrobacterium-mediated transformation (De Blaere et al. 1987. Meth. Enzymol. 143:277) and particle-accelerated or “gene gun” transformation technology (Klein et al. 1987. Nature (London) 327:70-73; U.S. Pat. No. 4,945,050, incorporated herein by reference). Additional transformation methods are disclosed below. Thus, isolated polynucleotides of the present invention can be incorporated into recombinant constructs, typically DNA constructs, capable of introduction into and replication in a host cell. Such a construct can be a vector that includes a replication system and sequences that are capable of transcription and translation of a polypeptide-encoding sequence in a given host cell. A number of vectors suitable for stable transfection of plant cells or for the establishment of transgenic plants have been described in, e.g., Pouwels et al. 1985. Supp. 1987. Cloning Vectors: A Laboratory Manual; Weissbach and Weissbach. 1989. Methods for Plant Molecular Biology, Academic Press, New York; and Flevin et al. 1990. Plant Molecular Biology Manual, Kluwer Academic Publishers, Boston. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

As used herein, the term “express” or “expression” is defined to mean transcription alone. The regulatory elements are operably linked to the coding sequence of the WEEP gene such that the regulatory element is capable of controlling expression of the WEEP gene. “Altered levels” or “altered expression” refers to the production of gene product(s) in transgenic organisms in amounts or proportions that differ from that of normal or non-transformed organisms.

In one embodiment, the polynucleotide encoding WEEP (SEQ ID NO: 2), the reverse complement of WEEP, or a portion thereof (e.g., SEQ ID NO: 4), operably linked to one or two appropriate promoters, can be stably inserted in a conventional manner into the genome (cytoplasmic genome or nucleic genome) of a single plant cell, and the genetically altered plant cell can be used in a conventional manner to produce a genetically altered plant that produces the dsRNA of this invention. In this regard, a disarmed Ti-plasmid, containing the polynucleotide of this invention, in Agrobacterium tumefaciens can be used to genetically alter the plant cell, and thereafter, a genetically altered plant can be regenerated from the genetically altered plant cell using the procedures described in the art, for example, in EP 0 116 718, EP 0 270 822, WO 84/02913 and EP 0 242 246. Plant regeneration from cultured protoplasts is described in Evans et al., Protoplasts Isolation and Culture, in Handbook of Plant Cell Culture, pp. 124-176, MacMillan Publishing Company, New York, 1983; and Binding, Regeneration of Plants, in Plant Protoplasts, pp. 21-73, CRC Press, Boca Raton, 1985. Regeneration can also be obtained from plant callus, explants, organs, or parts thereof. Such regeneration techniques are described generally in Klee, et al., Ann. Rev. of Plant Phys. 38:467-486 (1987). In one embodiment, the sense sequence and the antisense sequence in the dsRNA (and thus in the expression vector) are the same length so that they are complementary along their full-length.

Preferred Ti-plasmid vectors each contain the polynucleotides described herein between the border sequences, or at least located to the left of the right border sequence, of the T-DNA of the Ti-plasmid. Of course, other types of vectors can be used to transform the plant cell, using procedures such as direct gene transfer (as described, for example in EP 0 233 247), pollen mediated transformation (as described, for example in EP 0 270 356, WO 85/01856, and U.S. Pat. No. 4,684,611), plant RNA virus-mediated transformation (as described, for example in EP 0 067 553 and U.S. Pat. No. 4,407,956), liposome-mediated transformation (as described, for example in U.S. Pat. No. 4,536,475), and other methods such as the methods for transforming certain lines of corn (e.g., U.S. Pat. No. 6,140,553; Fromm, et al., Bio/Technology 8:833-839 (1990); Gordon-Kamm, et al., The Plant Cell 2:603-618 (1990) and rice (Shimamoto, et al., Nature 338:274-276 (1989); Datta et al., Bio/Technology 8:736-740 (1990)) and the method for transforming monocots generally (WO 92/09696). For cotton transformation, the method described in WO 2000/71733 can be used. For soybean transformation, reference is made to methods known in the art, e.g., Hinchee, et al. (Bio/Technology 6:915 (1988)) and Christou, et al. (Trends Biotechnology 8:145 (1990)) or the method of WO 00/42207.

The resulting genetically altered plant can be used in a conventional plant breeding scheme to produce more genetically altered plants with the same characteristics or to introduce the polynucleotide encoding WEEP (sense and/or anti-sense) into other varieties of the same or related plant species. Seeds, which are obtained from the genetically altered plants, contain an expression vector containing WEEP (sense and/or anti-sense) as a stable genomic insert. Plants containing a dsRNA in accordance with the invention include plants having or derived from root stocks of plants containing an expression vector containing WEEP (sense and/or anti-sense).

For a genetically altered plant that produces dsRNA, one constructs an expression vector or cassette (made from DNA) that encodes, at a minimum, a first promoter and the dsRNA sequence of interest such that the promoter sequence is 5′ (upstream) to and operably linked to the dsRNA sequence. The expression vector or cassette may optionally contain a second promoter (same as or different from the first promoter) upstream and operably linked to the reverse complementary sequence of the dsRNA sequence such that two strands of RNA that are complementary to each other can be produced. Alternatively, the expression vector or cassette can contain one promoter operably linked to both the dsRNA sequence (sense strand) in question and the complement or reverse complement of the dsRNA sequence (anti-sense strand) in question, such that the transcribed RNA can bend on itself and the two desires sequences can anneal. Alternatively, a second expression vector or cassette (made from DNA) can encode, at a minimum, a second promoter (same as or different from the promoter) operably linked to the reverse complementary sequence of the dsRNA such that two strands of complementary RNA can be produced in the plant. The expression vector(s) or cassette(s) is/are inserted in a plant cell genome (nuclear or cytoplasmic). The promoter(s) used should be a promoter(s) that is/are active in a plant and is/are heterologous to WEEP (not normally driving the transcription of RNA of genomic WEEP). Of course, the expression vector or cassette can have other transcription regulatory elements, such as enhancers, terminators, etc.

Promoters (and more specifically, heterologous promoters for WEEP or the reverse complement of WEEP) that are active in plants are well-known in the field. Such promoters can be constitutive, inducible, and/or tissue-specific. Non-limiting examples of constitutive plant promoters include 35S promoters of the cauliflower mosaic virus (CaMV) (e.g., of isolates CM 1841 (Gardner, et al., Nucleic Acids Research 9:2871-2887 (1981)), CabbB-S (Franck, et al., Cell 21:285-294 (1980)) and CabbB-JI (Hull and Howell, Virology 86:482-493 (1987))), ubiquitin promoter (e.g., the maize ubiquitin promoter of Christensen, et al., Plant Mol. Biol. 18:675-689 (1992)), gos2 promoter (de Pater, et al., The Plant J. 2:834-844 (1992)), emu promoter (Last, et al., Theor. AppL Genet. 81:581-588 (1990)), actin promoter (see, e.g., An, et al, The Plant J. 10:107 (1996)) and Zhang, et al., The Plant Cell 3:1155-1165 (1991)); Cassava vein mosaic virus promoters (see, e.g., WO 97/48819 and Verdaguer, et al., Plant Mol. Biol. 37:1055-1067 (1998)), the pPLEX series of promoters from Subterranean Clover Stunt Virus (WO 96/06932, particularly the S4 or S7 promoter), alcohol dehydrogenase promoter (e.g., pAdh1S (GenBank accession numbers X04049, X00581)), and the TR1′ promoter and the TR2′ promoter which drive the expression of the 1′ and 2′ genes, respectively, of the T-DNA (Velten, et al., EMBO J. 3:2723-2730 (1984)). Tissue-specific promoters are promoters that direct a higher level of transcriptional expression in some cells or tissues of the plant than in other cells or tissue. Non-limiting examples of tissue-specific promoters include the phosphoenolpyruvate carboxylase (PEP or PPC1) promoter (Pathirana, et al., Plant J. 12:293-304 (1997), and Kausch, et al., Plant Mol. Biol. 45 (1):1-15 (2001)), chlorophyll A/B binding protein (CAB) promoter (Bansal, et al., Proc. Natl. Acad. Sci. USA 89 (8):3654-8 (1992)), small subunit of ribulose-1,5-bisphosphate carboxylase (ssRBCS) promoter (Bansal, et al., Proc. Natl. Acad. Sci. USA 89 (8):3654-8 (1992)), senescence activated promoter (SEE1) (Robson, et al., Plant Biotechnol. J. 2 (2):101-12 (2004)), and sorghum leaf primoridia specific promoter (RS2) (GenBank Accession No. E1979305.1). These promoters (PPC1, CAB, ssRBCS, SSE1, and RS2) are all active in the aerial part of a plant. Further, the PPC1 promoter is a strong promoter for expression in vascular tissue. Some examples of phloem specific promoters are the sucrose synthase-1 promoters (CsSUS1p and CsSUS1p-2) (Singer et al., Planta 234:623-637 (2011)) and the phloem protein-2 promoter (CsPP2) (Miyata et al., Plant Cell Report 31 (11):2005-2013 (2012)) from Citrus sinensis. Alternatively, a plant-expressible promoter can also be a wound-inducible promoter, such as the promoter of the pea cell wall invertase gene (Zhang, et al, Plant Physiol. 112:1111-1117 (1996)).

Other types of RNA polymerase promoters that can be used are promoters from microorganisms, such as, but not limited to the bacteriophage T7 RNA polymerase promoter, yeast Galactose (GAL1) promoter, yeast glyceraldehyde-3-phosphate dehydrogenase (GAP) promoter, yeast Alcohol Oxidase (AOX) promoter.

One aspect of this invention is that one can cause a woody shrub or tree to have the weeping phenotype (compared to the phenotype of the wild-type woody shrub or tree) by reducing the amount of functional WEEP protein present in the genetically altered woody shrub or tree (compared to the amount of functional WEEP present in wild-type woody shrub or tree). Thus, in one embodiment, the genetically altered woody shrub or tree can have a null mutation in WEEP. A null mutation is a mutation within the target gene (WEEP) such that (i) no protein is produced, (ii) a truncated protein is produced which has reduced or no functionality, and (iii) a full-length protein is produced which has reduced or no functionality compared to the functionality of the non-mutated protein. A null mutation can result from changing a codon encoding an amino acid (in the wild-type woody shrub or tree) to a stop codon (in the genetically altered woody shrub or tree). See Table 1 supra for the sequence of stop codons. Another type of null mutation results from one or more altering splice site codons so that the protein produced (if any is produced) has reduced or no functionality. A third type of null mutation is the removal of most or all of the DNA sequence encoding a gene. One method for generating such a null mutation is by transforming the plant with a plasmid containing 5′ sequence and 3′ sequence of the gene and allowing a cross-over event to occur, thereby excising the DNA from the plant's genome that is between the plasmid's 5′ sequence and 3′ sequence. In addition, one can alter the sequence of the ribosome binding site upstream of the target protein such that ribosomes do not bind to the mRNA and translate the mRNA into protein. In one embodiment of this invention, a mutated genomic WEEP having the sequence of SEQ ID NO: 36 is expressly excluded from the sequence of a WEEP mutation (null or deletion or other type of mutation) in a genetically altered plant having the weeping phenotype.

Various methods exist to create a null mutation. These methods are well-known to one of ordinary skill in the art. Two such methods involves using a chemical mutagens (such as ethyl methanesulfonate (EMS)) or radiation (UV or proton, for example) to generate genetic mutations in plant cells and/or germplasm. Alternatively, one can use TALEN or CRISPR-Cas9 to mutate the sequence of the target gene (WEEP) such that a null mutation is generated. One of ordinary skill in the art can also use targeted cleavage events to induce targeted mutagenesis, induce targeted deletions of cellular DNA sequences, and facilitate targeted recombination and integration at a predetermined chromosomal locations to generate one or more of the null mutations discussed above or to reduce the mutated protein's functionality. Nucleotide editing techniques are well-known and described in Urnov, et al., Nature 435 (7042):646-51 (2010); U.S. Patent Publications 2003/0232410, 2005/0208489, 2005/0026157, 2005/0064474, 2006/0188987, 2009/0263900, 2009/0117617, 2010/0047805, 2011/0207221, 2011/0301073, 2011/089775, 2011/0239315, and 2011/0145940; and International Publication WO 2007/014275, the disclosures of which are incorporated by reference in their entireties for all purposes. Cleavage can occur through the use of specific nucleases such as engineered zinc finger nucleases (ZFN), transcription-activator like effector nucleases (TALENs), or using the CRISPR/Cas9 system with an engineered crRNA/tracr RNA (‘single guide RNA’) to guide specific cleavage. U.S. Patent Publication 2008/0182332 describes the use of non-canonical zinc finger nucleases (ZFNs) for targeted modification of plant genomes; U.S. Patent Publication 2009/0205083 describes ZFN-mediated targeted modification of a plant EPSPS locus; U.S. Patent Publication 2010/0199389 describes targeted modification of a plant Zp15 locus and U.S. Patent Publication No. 20110167521 describes targeted modification of plant genes involved in fatty acid biosynthesis. In addition, Moehle, et al, Proc. Natl. Acad. Sci. USA 104 (9):3055-3060 (2007) describes using designed ZFNs for targeted gene addition at a specified locus. U.S. Patent Publication 2011/0041195 describes methods of making homozygous diploid organisms. Information on CRISPR/Cas9 system can be found, e.g., at en.wikipedia.org/wiki/CRISPR; neb.com/tools-and-resources/feature-articles/crispr-cas9-and-targeted-genome-editing-a-new-era-in-molecular-biology; and Cong, et al., Science, 339:819-823 (2013). Sigma-Aldrich (St. Louis, Mo.) and Origene Technologies, Inc. (Rockville, Md.) are among the companies that sell CRISPR/Cas9 kits. Any RNA-guided DNA endonuclease that works with CRISPR can be used instead of Cas9.

After using any of these various methods to induce genetic alterations in a cell's genome, one can induce the treated cells to grow into plants and then screen the plants using the methods described herein for WEEP having reduced or no functionality, and/or for reduced amounts of WEEP or no WEEP (via reduction in gene expression and/or mRNA translation and/or other mechanism), and/or for weeping phenotype (compared to amounts present in wild-type plants). Thus, another embodiment of this invention is the generation of genetically altered woody shrubs and/or trees having a null mutation in Weep such that the genetically altered woody shrub and/or tree has the weeping phenotype compared to the phenotype of the wild-type woody shrub and/or tree.

The term “plant” includes whole plants, plant organs, progeny of whole plants or plant organs, embryos, somatic embryos, embryo-like structures, protocorms, protocorm-like bodies (PLBs), and suspensions of plant cells. Plant organs comprise, e.g., shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, trichomes and the like). The class of plants that can be used in the method of the invention is generally as broad as the class of higher and lower plants amenable to the molecular biology and plant breeding techniques described herein, specifically angiosperms (monocotyledonous (monocots) and dicotyledonous (dicots) plants). It includes plants of a variety of ploidy levels, including aneuploid, polyploid, diploid, haploid and hemizygous. The genetically altered plants described herein include eudicots, and in another embodiment, woody shrubs and trees. In another embodiment that eudicot is a Prunus cultivar, including but not limited to, Prunus persica (peach), Prunus domestica (plum), Prunus avium (cherry), Prunus salicina (Japanese plum) and Prunus armeniaca (apricot).

As used herein, the term “plant” includes reference to whole plants, plant organs (e.g., leaves, stems, roots, etc.), seeds, plant cells, and progeny of same. Parts of transgenic plants are to be understood within the scope of the invention to comprise, for example, plant cells, protoplasts, tissues, callus, embryos as well as flowers, stems, fruits, leaves, roots originating in transgenic plants or their progeny previously transformed with a DNA molecule of the invention and therefore consisting at least in part of transgenic cells, are also an object of the present invention.

As used herein, the term “plant cell” includes, without limitation, seeds suspension cultures, embryos, meristematic regions, callus tissue, leaves, roots, shoots, gametophytes, sporophytes, pollen, and microspores. The class of plants that can be used in the methods of the invention is generally as broad as the class of higher plants amenable to transformation techniques, including both monocotyledonous and dicotyledonous plants.

Many techniques involving molecular biology discussed herein are well-known to one of ordinary skill in the art and are described in, e.g., Green and Sambrook, Molecular Cloning, A Laboratory Manual 4th ed. 2012, Cold Spring Harbor Laboratory; Ausubel et al. (eds.), Current Protocols in Molecular Biology, 1994-current, John Wiley & Sons; and Kriegler, Gene Transfer and Expression: A Laboratory Manual (1993). Unless otherwise noted, technical terms are used according to conventional usage. Definitions of common terms in molecular biology maybe found in e.g., Benjamin Lewin, Genes IX, Oxford University Press, 2007 (ISBN 0763740632); Krebs, et al. (eds.), The Encyclopedia of Molecular Biology, Blackwell Science Ltd., 1994 (ISBN 0-632-02182-9); and Robert A. Meyers (ed.), Molecular Biology and Biotechnology: a Comprehensive Desk Reference, VCH Publishers, Inc., 1995 (ISBN 1-56081-569-8).

The terms “approximately” and “about” refer to a quantity, level, value or amount that varies by as much as 30% in one embodiment, or in another embodiment by as much as 20%, and in a third embodiment by as much as 10% to a reference quantity, level, value or amount. As used herein, the singular form “a”, “an”, and “the” include plural references unless the context clearly dictates otherwise. For example, the term “a bacterium” includes both a single bacterium and a plurality of bacteria.

The term “nucleic acid consisting essentially of”, “polynucleotide consisting essentially of”, and “RNA consisting essentially of”, and grammatical variations thereof, means a polynucleotide that differs from a reference nucleic acid sequence by 20 or fewer nucleotides and also perform the function of the reference polynucleotide sequence. Such variants include sequences which are shorter or longer than the reference nucleic acid sequence, have different residues at particular positions, or a combination thereof.

Having now generally described this invention, the same will be better understood by reference to certain specific examples, which are included herein only to further illustrate the invention and are not intended to limit the scope of the invention as defined by the claims.

### Example 1. Isolation and Identification of Ppa013325

The peach population (planted in the field in June 2009) used for the p-nome gene mapping was generated from a 2008 self-pollination of tree Kv050168 at the USDA Agricultural Research Service Appalachian Fruit Research Station (AFRS) in Kearneysville, W. Va. This population, which was grown in the field at AFRS, had Mendelian recessive segregation for the weeping phenotype. Kv050168 originated from a cross between AFRS Kv010095 (a weeping red-leafed Baily peach) and Kikomo D (a chrysanthemum flowered peach from an open-pollination of Kikomo seed sent from Japan and released from USDA APHIS quarantine in 2000. AFRS tree Kv010095 was the progeny of an open pollination of Kv981549, which was the progeny of a cross between Bailey and Kv931777. Kv931777 was a seedling from a cross between Bailey and tree number 14DR60. The weeping phenotype is believed to have originated from 14DR60. The additional AFRS population that segregates weeping (and was genotyped and used for RNA sequencing) was from a self-pollination of Kv991636 in 2002. KV991636 originated from a cross between Kv93065 (a pillar tree) and ‘weeping white’ pollen. The ‘weeping white’ peach tree was collected from a tree in New Jersey in or before 1986.

The peach populations in France used by the INRA Genetique et Amelioration des Fruites et Legumes (GAFL) for phenotyping and genotyping are described as follows. An F₂ mapping population named WP² was used to map the pl locus. WP² (n=336) was obtained in 2010 from the self-pollination of a single tree (n° 3) derived from a controlled cross between the peach varieties Weeping Flower Peach (clone 52678) and Pamirskij 5 (clone S6146). Introduced to the INRA (France) in 1961from Clemson University (South Carolina—USA), S2678 is an ornamental peach tree studied by Monet et al. (supra) for its weeping growth habit (plpl). 56146 is a peach rootstock derived from seeds sent by the Nikita Botanical Garden of Yalta (Pascal etal. 2010. HortScience 45:150-152), here chosen for its standard tree habit (PlPl). Planted in orchard conditions at Experimental Facilities of ‘Saint Maurice’ (INRA-UGAFL-France), WP² had Mendelian recessive segregation for the weeping phenotype. Phenotyping of WP² individuals for weeping growth habit trait (pl) thus was scored in accordance with a Mendelian character (weeping/standard), as already performed (Monet et al., supra; Chaparro et al., supra).

DNA for genomic sequencing and genotyping was extracted using the Omega Bio-Tek (Norcross, Ga., USA) EZNA SQ Plant DNA extraction kit with the RNAse step (Cat no. D3095-01). DNA concentrations for sequencing were calculated using the Molecular Probes QuantiT™ PicoGreen® dsDNA Assay (Life Technologies, Frederick, Md., USA, Cat no. P11496).

A whole-genome sequencing method was employed to map the recessive locus responsible for the weeping peach growth habit, which is visible within one year of growth. This sequencing method, named pnome (for pooled genome), was previously described and successfully used to identify genes associated with peach pillar and brachytic dwarf architectures (Dardick, et al., 2013. Plant J. 75:618-630; Hollender, et al., 2016. New Phytol. 210 (1):227-239). The pnomes strategy is based on sequencing a population(s) of segregating individuals pooled by a specific trait(s). In theory, the linkage of individual polymorphisms to a trait of interest should be measurable by calculating the abundance of each polymorphism within a given pnome assembled against a reference genome. Tightly linked polymorphisms should occur at high frequency in the pnome containing the trait while those same polymorphisms should be rare or absent in the pnome lacking the trait, and vice versa. Consequently, when graphed by nucleotide position, the data should produce a bell-shaped curve delineating the location of the trait.

DNA from 55 standard trees from the Kv050168 population and 19 weeping trees from a four-year-old segregating population were pooled by architecture type with each pool containing the same amount of DNA from each tree. The DNA pools, with final concentrations between 2.5 μg and 4 μg, were sent to the genomics resources core facility at Weill Cornell Medical College (New York, N.Y., USA) and 100 bp paired-end sequencing was performed at Weill Cornell Medical College (New York, N.Y.) with an Illumina HiSeq 2000 (San Diego, Calif.) with each library in a separate lane. Weeping library generated 355,011,274 raw reads and the standard generated 367,203,548 raw reads. Raw reads were imported into CLC Genomics Workbench version 6.1 (Qiagen, Gaithersburg, Md.) and trimmed based on quality (with an ambiguity limit of two nucleotides and a quality limit of 0.05) and reads <75 nucleotides in length were removed. The remaining 354,634,975 weeping and 366,694,736 standard reads were aligned to the peach genome (version 1.0 scaffolds) (Verde, et al., 2013. Nat. Publ. Gr. 45 (5):487-494). Next, the probabilistic variant detection function in Workbench was performed on both alignments with the following settings: ignore non-specific matches; ignore broken pairs; minimum coverage 25; variant probability 90; requires presence in both forward and reverse reads; maximum expected variants 2. The weeping pool sequencing reads contained 1,156,590 variants, while the standard pool contained 1,221,826.

The sequences from each pool were assembled to the peach genome (version 1.0) and variants including Single Nucleotide Polymorphisms (SNPs) and insertions or deletions (IN/DELs) that existed between the published genome and the pools were identified (Verde, et al., 2013. Nat. Genet. 45 (5):487-494). 644,488 variants were present in the standard pool sequences and 615,035 variants were detected in the weeping pool sequences.

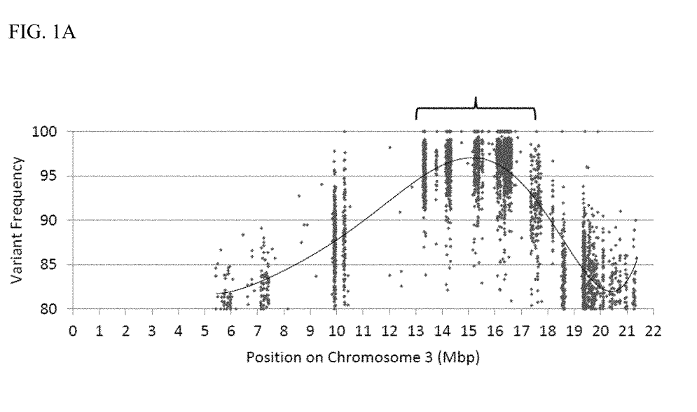

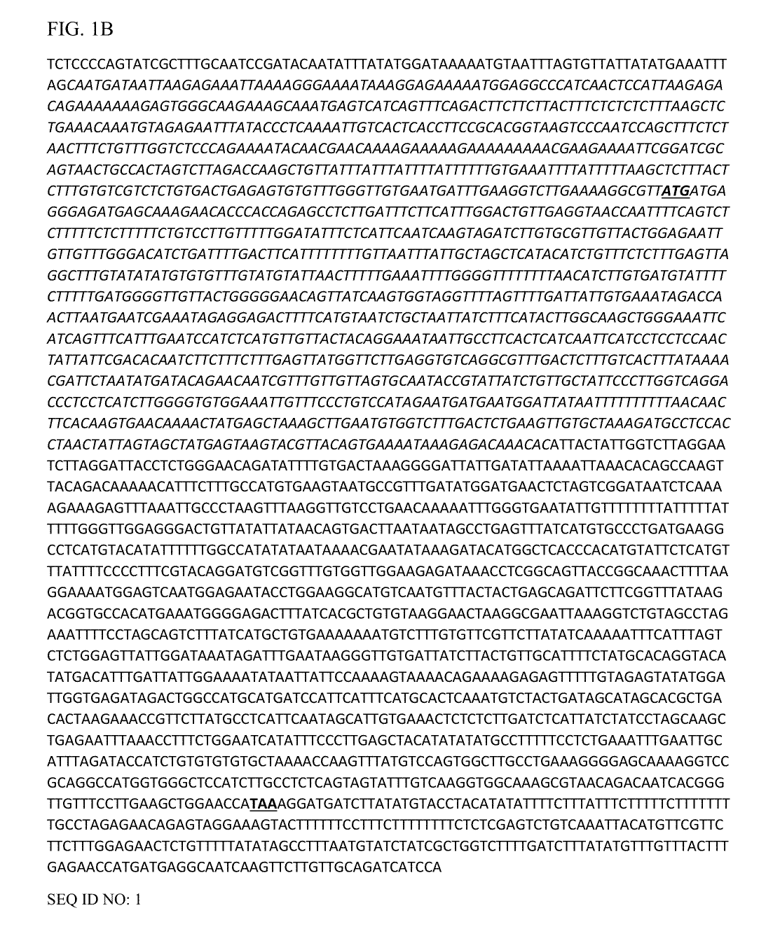

The weeping pool variant list was manually filtered to remove variants that were infrequently present as well as ones with that had high frequencies in the standard pool. Variant data was exported into Microsoft Excel® (Redmond, Wash.) spreadsheet for manual filtering. All variants in the weeping pool with a frequency less than 80% were removed, as were variants in the weeping pool with a forward/reverse balance less than 10%, and variants with coverage greater than 500 were removed. Next, the variants present in the standard pool with frequencies greater than 45% and less than 20% were removed from the weeping variant list. The remaining variants were graphed by frequency of occurrence over chromosome position. 84% of all variants mapped to chromosome 3, (3,896 variants) and produced a bell curve indicating the region of linkage (FIG. 1A). The peak of the curve spanned a 2 Mbp chromosomal region (between ˜14.2 Mbp and ˜16.2 MBp) and contained 256 predicted genes and 173 coding region changes but no obvious candidate gene could be identified based on amino acid changes (FIG. 1A).

To narrow down the candidate list, next-generation Illumina RNA sequencing (RNAseq) was performed on total RNA from one standard and one weeping tree from the mapping population in an attempt to identify differentially expressed genes located in the mapped region. Total RNA was isolated from ˜3-6 cm of actively growing shoot tips (with leaves removed) from one weeping and one standard peach tree from the mapping population. RNA was extracted using the SQ Total RNA kit (Omega Bio-tek, Norcross, Ga.) with 2% PCP added to the RCL buffer followed by a DNAse step using Turbo DNAfree™ Kit (ThermoFisher Scientific, Waltham, Mass.) prior to phenol/chloroform purification. Approximately 3 μg of RNA was sent to the genomics resources core facility at Weill Cornell Medical College (New York, N.Y.) where RNA TruSeq 50 bp unpaired libraries were prepared for each and sequenced in the same lane using an Illumina HiSeq 2000. Raw sequencing data was uploaded to CLC Genomics Workbench (Qiagen, Gaithersburg, Md.) and trimmed based on quality (setting at 0.05) and ambiguity (max. 2 ambiguous nucleotides allowed). The remaining 86,592,113 reads from the weeping tree and 77,747,375 reads from the standard tree were aligned to the peach genome v1.0 using CLC Genomics Workbench with the following parameters: additional upstream and downstream bases=500; max number of mismatches=2; minimum length fraction=0.9, minimum similarity fraction=0.8; unspecific match limit=10; No strand specific assembly; strand=forward; no exon discovery; minimum exon coverage fraction=0.2; minimum number of reads=10; expression value=RPKM. Differential expression was determined using the Transcriptomics Analysis function in CLC Genomics Workbench.

Ppa013325, a gene in the center of this region (at 15.6 Mbp; on the minus strand between position 15,603,515 and 15,601,388) was expressed 127 fold less in the weeping tree compared to the standard tree. No other gene in this region had an expression increase or decrease greater than 6.4 fold. Inspection of the RNAseq assembly revealed that the small number of reads derived from the weeping pool spanned only the 3′ region of the gene. Subsequent examination of the genomic sequence alignments from both the weeping and standard p-nome pools revealed that the weeping alignment contained numerous broken paired-end reads and very minimal coverage, denoting a ˜1,374 bp deletion spanning part of the promoter and the 5′ end of Ppa013325. Additionally, RNAseq reads from the standard tree spanned all of the prediction exons for Ppa013325 while the reads from the weeping tree only spanned the 5′ region. This difference suggested the presence of either deletion or insertion in the 5′ region of Ppa013325 in the weeping trees, which would prevent transcription of the full gene. Subsequent examination of the genomic sequence alignments from both pools revealed that the weeping alignment contained numerous broken 100 bp reads and minimal coverage in a ˜1800 bp region in the 5′ end of Ppa013325. FIG. 1B shows the genomic sequence spanning from 15,604,111 to 15,601,132 (SEQ ID NO: 1). The italicized sequences indicate genomic sequences absent in the naturally occurring weeping trees. The ATG start codon and TAA stop codon are underlined. The sequence of the naturally occurring WEEP deletion mutation (which is SEQ ID NO: 1 without the italicized region) is SEQ ID NO: 36.

To further investigate if the truncated Ppa013325 allele was associated with the weeping phenotype, fine mapping was performed using 453 peach trees originating from 4 peach populations (3 discrete lineages) that segregated for weeping. First, 125 trees located at the Appalachian Fruit Research Station, Kearneysville, W. Va. were tested including 71 individuals from the p-nome population, 42 weeping trees from a related F2 population, and 12 trees from a segregating population with a different weeping lineage. For this analysis DNA was re-extracted from the mapping population trees. High Resolution Melt (HRM) was performed using MeltDoctor™ HRM Master Mix (Thermo Fisher Scientific, Waltham, Mass.), according to the manufacturer's protocol, and the reactions were run on a ViiA™ Real-Time PCR System instrument (Thermo Fisher Scientific, Waltham, Mass.) as described in Hollender, et al., supra. For genotyping using traditional PCR, the following primers, which spanned the deletion, were used to detect the weeping mutants: PpWEEP-Del-genotype-F2 (5′-GATTGTGAAGGACACGTAGCT-3′; forward; SEQ ID NO: 5) and PpWEEP-Del-genotype-R2 (5′-TGTCTGTAACTTGGCTGTGTTTA-3′; reverse; SEQ ID NO: 6) using an annealing temperature of 63° C. to produce an ˜300 bp band. To detect the wild type allele, the following primers, which amplify a sequence within the deletion, were used: Pp WEEP Del internal F2 (5′-TGTTGTTTGGGACATCTGAT-3′; forward; SEQ ID NO: 7) and Pp WEEP Del internal R2 (5′-AGCAGATTACATGAAAAGTCTCCT-3′; reverse; SEQ ID NO: 8) with an annealing temperature of 56° C. to produce a 279 bp amplicon. HRM results scored all weeping trees as homozygous for markers on both sides of the deletion, while the standard trees were either heterozygous or homozygous wild type for those markers. SNP markers flanking Ppa013325 at various intervals confirmed the locus and reduced the interval to a 502Kb region (position 15,537,956 and 16,040,400).

Next, 328 trees comprising a segregating population at INRA Unité de Génétique et Amélioration des Fruits et Légumes (UGAFL), France were used for further fine mapping. First, a SNP linkage map using 91 segregating individuals was created using the International Peach SNP Consortium (IPSC) 9K peach SNP array v1 (Illumina Inc. San Diego, Calif., USA) according to the protocol set forth in Verde, et al., 2012. PLoS One 7 (4):e35668. DNA was diluted to 50 ng/pl and sent to the IASMA Research and Innovation Centre (San Michele all'Adige, Italy) for genotyping. The assays were performed following the manufacturer's recommendations. SNP genotypes were scored with the Genotyping Module of GenomeStudio Data Analysis software (Illumina Inc., San Diego, Calif.), using a GenCall threshold of 0.15. SNPs with GenTrain scores<0.6 were used to clean up the data file. SNPs showing severe segregation distortion (X² test, p<10⁻⁶) and more than 1% of missing data were excluded. Linkage mapping at UGAFL was performed as follows. For the WP² map, linkage analyzes were performed using JoinMap v4.1. See, Van Ooijen, J. W., 2006. Kyazma B V, Wageningen 33:10-1371. The recombination fraction value was set at 0.4 and the initial minimum LOD score threshold at 3. Recombination frequencies were converted into marker distances using the Kosambi mapping function. See, Kosambi, D. D., 1943. Ann. Eugen. 12:172-175. At first, the quality of markers was checked, and those with a high number of missing/conflicting (repetitions) data were discarded. In a second step, “non-useful” markers were discarded like those monomorphic in the population or those presenting a very high degree of segregation distortion (X² test). Only polymorphic SNPs homozygous in both parents (AAxBB) were used to construct the genetic map of WP². Map figure was drawn using MapChart software (Voorrips, R. E., 2002. J. Hered. 93 (1):77-78), and the order and distribution of markers on the genetic map were compared to their positions in peach sequence v1.0. These results confirmed the position of the weeping locus (pl) to chr3 between position 15,203,630 and 16,351,739.

In order to rule out the possibility that a separate, tightly linked polymorphism could account for the weeping phenotype, the entire ˜435 kb locus was further analyzed. A total of 56 predicted genes were annotated within the locus including Ppa013325. A total of 10 sequence variants were present in addition to the Ppa013325 deletion. Eight were SNPs found in intergenic regions. The other 2 variants were single base IN/DELs (+T and −A, respectively) within homopolymeric intron sequences of Ppa007938 and Ppa006798. Given that neither of these genes were differentially expressed in the RNAseq data and showed no differences in coding or splicing, they were deemed unlikely candidates for the pl allele. Based on the combined mapping data and lack of alternative gene variants within the mapped region, Ppa013325 was designated as WEEP.

The cds of normal Ppa013325 is in SEQ ID NO: 2. The amino acid sequence of the encoded protein is in SEQ ID NO: 3. The sequence of the naturally occurring WEEP deletion mutation is SEQ ID NO: 36.

### Example 2. Protein Alignments and Phylogenetic Tree Construction

Proteins alignments were generated using Muscle v3.8.425 (Edgar, R. C., 2004. Nucleic Acids Res 32 (5):1792-1797). Maximum likelihood phylogenetic tree was generated in CLC Genomics Workbench using the UPGMA algorithm with Kimura distance measurements and 100 bootstrap replicates.

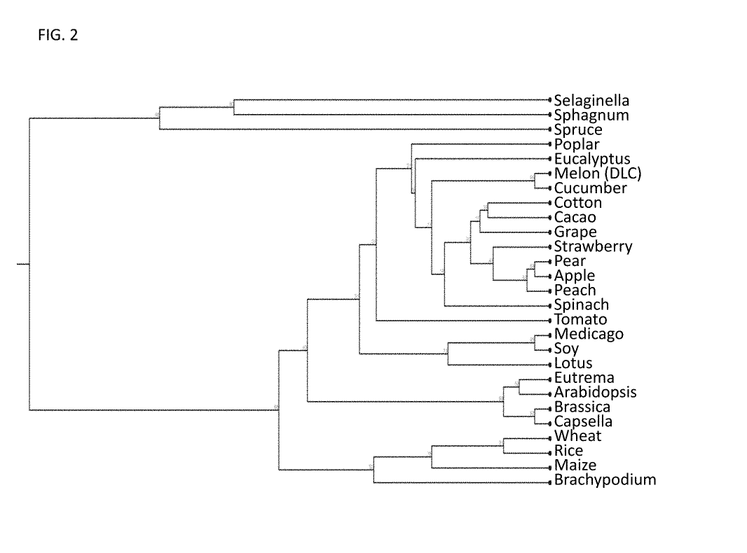

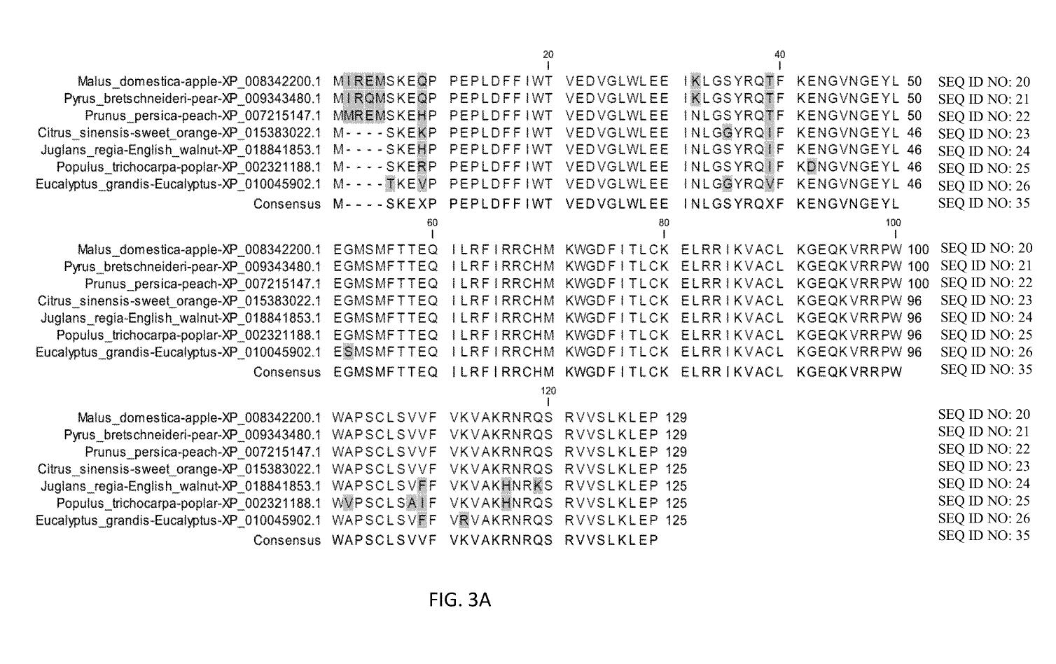

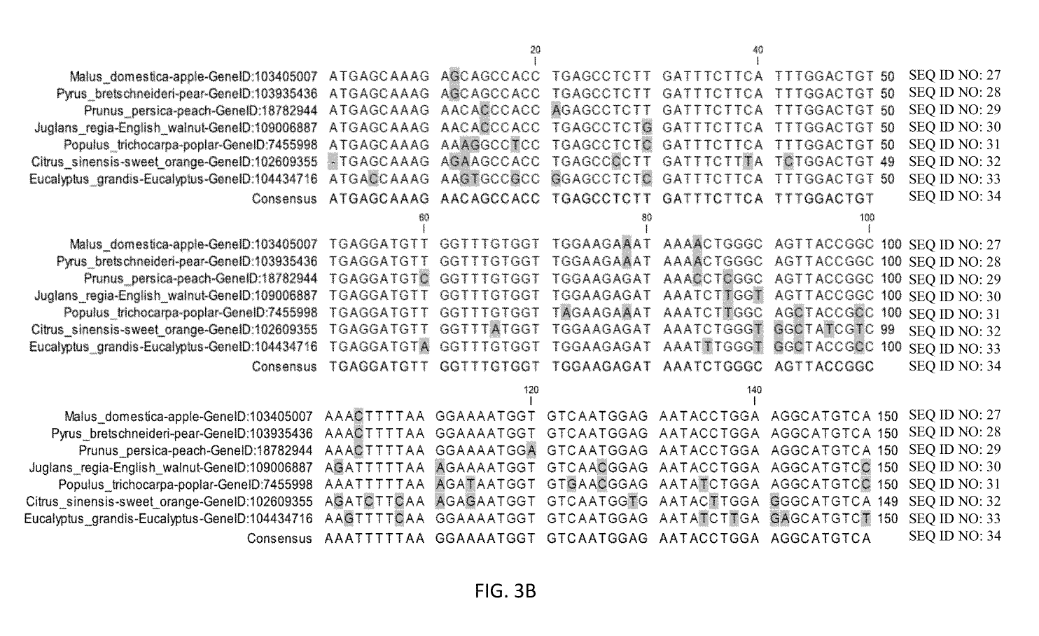

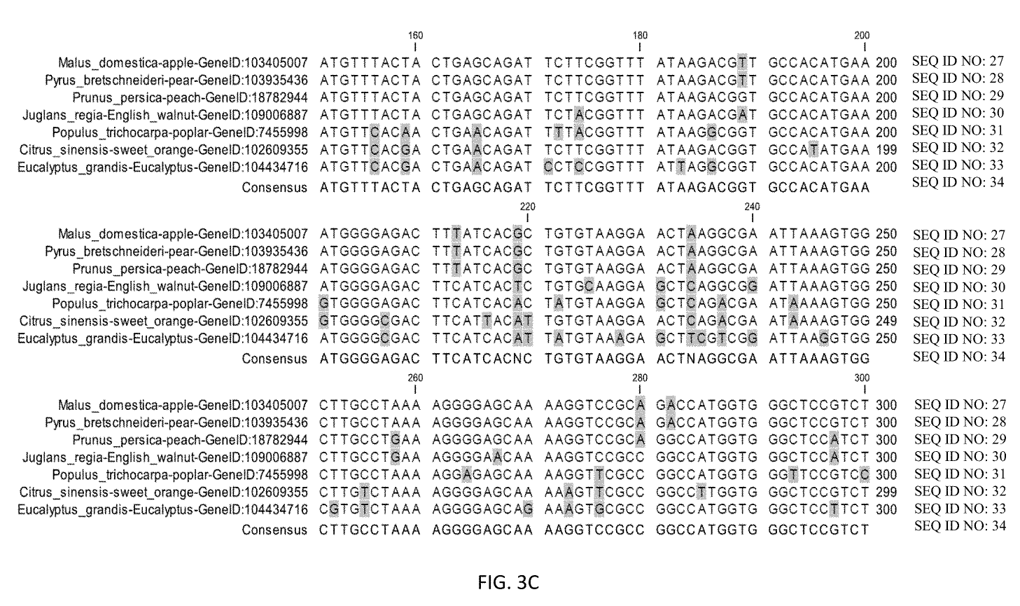

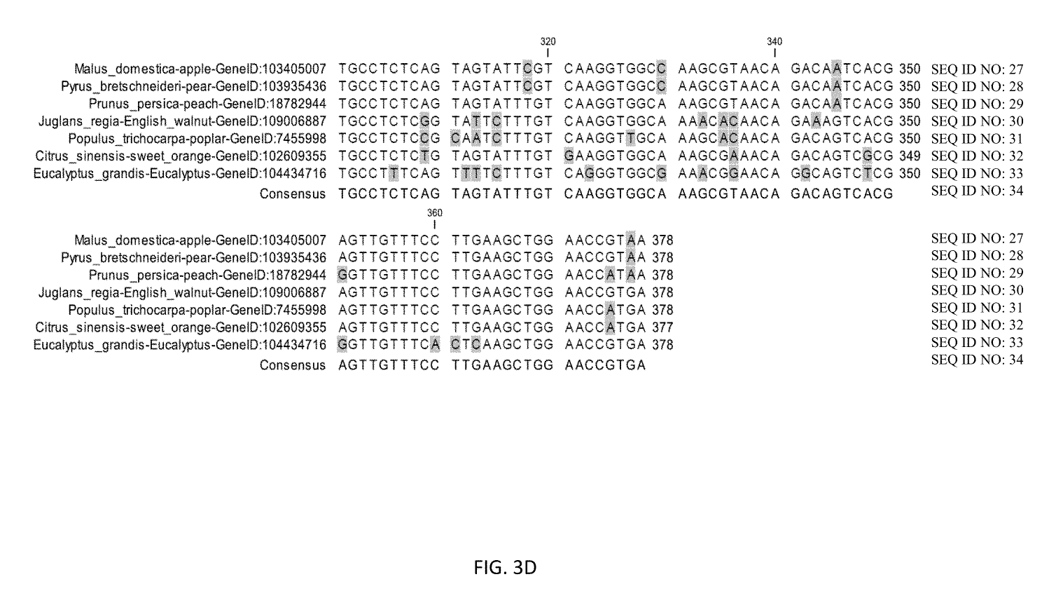

WEEP is an ancient and highly conserved gene encoding a sterile alpha motif (SAM) domain protein and is typically present as a single copy gene. See FIG. 2. WEEP homologues were not found in the moss genome Physcomitrella patens or Chlorophyte genomes but are present in the moss Sphagnum phallax and the genome of the lycophyte Selaginella moellendorffii. Phylogenetic analyses revealed known plant species relationships among angiosperms with the exception of the Brassicaceae family, which contained five conserved amino acid changes and formed an out-group from other eudicots. Four of these substitutions were located within the SAM domain FIG. 3A shows the amino acid sequence alignment for WEEP proteins in the following eudocots: Malus domestica (SEQ ID NO: 20; protein ID XP_008342200.1), Pyrus bretschneideri (SEQ ID NO: 21, protein ID XP_009343480.1), Prunus persica (SEQ ID NO: 22, protein ID XP_007215147.1), Citrus sinensis (SEQ ID NO: 23, protein ID XP_015383022.1), Juglans regia (SEQ ID NO: 24, protein ID XP_018841853.1), Populus trichocarpa (SEQ ID NO: 25, protein ID XP_002321188.1), and Eucalyptus grandis (SEQ ID NO: 26, protein ID XP_010045902.1). The consensus amino acid sequence is SEQ ID NO: 35. FIGS. 3B, 3C, and 3D show the DNA sequence alignment for WEEP gene in the following eudicots: Malus domestica (SEQ ID NO: 27; gene ID 103405007), Pyrus bretschneideri (SEQ ID NO: 28, gene ID 103935436), Prunus persica (SEQ ID NO: 29, gene ID 18782944), Juglans regia (SEQ ID NO: 30, gene ID 109006887), Populus trichocarpa (SEQ ID NO: 31, gene ID 7455998), Citrus sinensis (SEQ ID NO: 32, gene ID 102609355), and Eucalyptus grandis (SEQ ID NO: 33, gene ID 104434716). The consensus DNA sequence is SEQ ID NO: 34. Because WEEP is highly conserved sequence amongst eudicot trees, one can generate an eudicot plant/tree with the weeping phenotype by genetically altering an eudicot plant/tree by (i) transforming a wild-type eudicot cell with an expression vector that contains a heterologous promoter operably linked to a polynucleotide that encodes at least 19 nucleotides of an eudicot WEEP, a linker, and a sequence complementary to the at least 19 nucleotides of an eudicot WEEP (thus encoding a dsRNA) (in one embodiment, the sense and antisense sequences in the dsRNA are of equal length), (ii) selecting at least one genetically altered eudicot cell that produce the dsRNA encoded by the expression vector, and (iii) inducing the selected genetically altered eudicot cell to grow into a genetically altered plant/tree that produces the dsRNA for WEEP, thereby reducing the amount of WEEP produced by the genetically altered eudicot plant/tree compared to the amount of functional WEEP produced by a wild-type eudicot plant/tree, and the small amount of WEEP in the genetically altered eudicot plant/tree causes a weeping phenotype. One can use this approach to make a weeping Malus domestica using dsRNA from SEQ ID NO: 27, Pyrus bretschneideri using dsRNA from SEQ ID NO: 28, Prunus persica using dsRNA from SEQ ID NO: 29, Juglans regia using dsRNA from SEQ ID NO: 30, Populus trichocarpa using dsRNA from SEQ ID NO: 31, Citrus sinensis using dsRNA from SEQ ID NO: 32, Eucalyptus grandis using dsRNA from SEQ ID NO: 33, and any other eudicot containing a WEEP gene that encodes a WEEP protein with 95% or greater identity to SEQ ID NO: 35. In an alternative embodiment, one can generate an eudicot tree with the weeping phenotype by (i) transforming a wild-type eudicot cell with an expression vector encoding an RNA-guided DNA endonuclease (such as, Cas9) and a sgRNA targeting WEEP, (ii) selected at least one genetically altered eudicot cell that produces the RNA-guided DNA endonuclease (such as, Cas9) and the sgRNA, and (iii) inducing the selected genetically altered eudicot cell to grow into a genetically altered eudicot plant/tree that contains a null mutation in WEEP that results in non-functional WEEP be produced by the genetically altered eudicot plant/tree which then causes the weeping phenotype. The sgRNA has a sequence that is obtained from the DNA that encodes a WEEP gene that encodes a WEEP protein with 95% or greater identity to SEQ ID NO: 35. Again, SEQ ID NOs: 27-33 are non-limiting examples of such WEEP genes that can be used. Examples 7 and 8, below, describe how one generates such genetically altered eudicot plant/trees having the WEEP phenotype. In one embodiment, sgRNA has the sequence of SEQ ID NOs: 16, 37, 38, 39, 40, or 41.

WEEP is primarily a single copy gene in species as ancient as the early vascular plant Selaginella moellendorfii. The high degree of protein conservation across vascular plants suggests WEEP may have a fundamental role in plant biology. Yet, the only clue to the molecular function of WEEP is the presence of a Sterile-Alpha-Motif (SAM) domain. Hundreds of SAM domain proteins have been identified throughout the protozoan, fungi, and animal kingdoms, and have functions ranging from kinases in signaling pathways, to scaffolding and RNA-binding proteins, to transcriptional activators or repressors (Ponting, C. P., 1995. Protein Sci. 4 (9):1928-1930; Schultz, et al., 1997. Protein Sci. 6:249-253, Qiao and Bowie, 2005. Sci. STKE re 7). The SAM domain itself contains a bundle of alpha helices and is a known protein and RNA binding domain (Qiao and Bowie, supra). In regards to protein interactions, the SAM domain enables homodimerization as well as heterodimerization with other SAM and non-SAM domain proteins (Ponting, supra; Shultz, et al., supra; Slaughter, et al., 2008. PLoS One 3: e1931-e1931; Qiao and Bowie, supra).

### Example 3. WEEP Tissue Expression in Wild-type Peach

WEEP expression was assessed in various vegetative peach tissues collected from field grown wild-type peach trees. To determine WEEP expression levels in peach, RNA was extracted from actively growing shoot apical and young leaf tissues from greenhouse grown plants. Peach epidermis, endodermis, and xylem tissues were dissected from trees from field-grown trees. Additional peach tissues were from greenhouse grown plants. The RNA extraction protocol used is the protocol described supra in Example 3. Again, WEEP expression levels in each tissue sample were determined by qPCR using SuperScript™ III Platinum™ SYBR™ Green One-Step qRT-PCR with Rox (Thermo Fischer Scientific, Waltham, Mass.) and run on an AB17900 (Thermo Fischer Scientific, Waltham, Mass.). 50 ng of total RNA were used for each reaction. Primers used for peach WEEP expression were Ppweep qPCR 1F (5′-CGTGATTGTCTGTTACGCTTTGC-3′, forward, SEQ ID NO: 14) and Ppweep qPCR 1R (5′-TCACGCTGTGTAAGGAACTAAGGC-3′, reverse; SEQ ID NO: 15). Relative expression values were determined from standard curves generated using known amounts of total RNA from standard peach trees. Expression in peach tissues was determined from between two and four biological replicates, each with three technical replicates.

WEEP expression was highest in shoots, particularly in the internode and nodal tissues and absent in dormant vegetative buds (FIG. 5A). Further dissection of stem tissues revealed WEEP was predominantly expressed in phloem/endodermal tissue (relative value ˜30) and to a lesser extent in xylem tissue (relative value<5) (FIG. 5B). WEEP expression was undetectable in epidermal tissues (FIG. 5B).

### Example 4. The Weeping Peach Did Not Respond to Gravistimulation

Phenotypic analyses were performed on weeping peach trees to help infer WEEP function. One-year-old standard and weeping peaches from the same population were gravistimulated by 90-degree rotation. The standard peach trees exhibit classic gravitropic responses. Gravistimulation resulted in a partial upward lifting vertical reorientation of primary shoot tips as well as the continuation of upward growth. Additionally, secondary shoots emerging from previously dormant buds on gravistimulated standard trees exhibited agravitropic (upward) growth. In contrast, after rotation, the shoot tips of weeping trees did not reorient. They continued apical growth in the downward direction. The lack of gravitropic response in weeping trees occurred no matter if the primary shoot was repositioned to have an upward or downward orientation. Additionally, newly emerged secondary shoots from weeping trees placed in either orientation arched downward and continued growth in the direction of the gravity vector.

The lack of a bending response to 90-degree rotations in weeping trees suggests impairments in gravity sensing, signaling, or the asymmetric growth needed to correct orientation disruptions. The rotations also illustrated that the weeping branch orientation was unrelated to initial positioning of vegetative buds, as both old and newly initiated shoots alike grew downwards no matter if the apical meristem and vegetative buds were oriented up or down.

### Example 5. Treatment with Growth Hormone Gibberellic Acid

It was previously reported that gibberellic acid (GA) treatment rescued the pendulous phenotypes in varieties of weeping peach and weeping cherry (Baba, et al., supra; Nakamura, et al., 1995, supra; and Nakamura, et al., 1994, supra). To test if the weeping peach variety responds the same way, 1000 ppm GA3 (0.01%) (in the form of ProGibb®; Valent BioSciences Corp., Libertyville, Ill.) was sprayed twice a week for one month on actively growing plants. The plants tested were two standard peach trees with weeping branches from prior bud grafts, three weeping trees, and three standard trees, all in 8″ pots in a greenhouse. An additional three weeping and three standard trees were sprayed with water. All trees were removed from cold dormancy ˜1 month prior to treatments.

In both standard and weeping trees, GA treatment promoted the release of vegetative buds from dormancy followed by rapid shoot growth. However, the resulting new growth on weeping shoots exhibited the characteristic arched downward growth, while the standard shoots had an upward orientation and growth trajectory. Thus, the peach weeping phenotype described here could not be rescued by GA application.

Pendulous weeping peach and cherry phenotypes were hypothesized to result from a lack of physical rigidity and delayed tension wood development on the upper side of branches in response to altered timing of gibberellic acid (GA) signaling (Nakamura, et al., 1994, supra; Baba et al., supra, Nakamura, et al., 1995, supra). Treatment of weeping peach and cherry varieties with GA resulted in upward shoot growth and GA treatment in cherry increased tension wood formation on the upper side of branches (Nakamura, et al., 1994, supra; Baba et al., supra, Nakamura, et al., 1995, supra). Additional GA abnormalities were also detected in weeping trees. Weeping cherry trees have higher levels of GA and greater expression of the GA biosynthetic gene gibberellin 3β-hydroxylase gene (Ps3ox) in branch elongation zones compared to upright cherry trees (Kobayashi et al. 1996. Bioscience; Sugano et al., supra). Lastly, Reches et al. (supra) found that the lower half of actively growing weeping mulberry (Morus alba Var. pendula) tree branches had significantly greater GA activity than the upper side. Despite the published data connecting GA to weeping tree phenotypes, GA treatment of our weeping peach variety however did not lead to upright shoot orientations. This lack of reversion was not due to GA insensitivity, as the treated trees did produce the typical/expected shoot elongation and release from dormancy. The branch orientation discrepancy between these studies may be due to a difference in the underlying cause behind the different weeping phenotypes or differences in the GA concentrations and application methods used. Alternatively, the branch reversion in the aforementioned studies may instead be an artifact of repeated application of high concentrations of GA and not relate to the underlying cause of the branch phenotypes.

### Example 6. Acidic Hormone Analysis

Because GA treatment did not promote upright growth in the weeping peach, the potential roles of other plant hormones in the weeping phenotype was investigated. Asymmetrical concentrations of auxin have long been associated with gravitropic bending responses (Yoshida, et al., 1999. J. Wood Sci. 45:368-372). In addition, abscisic acid (ABA) has been shown to have an opposite role of auxin in gravitropism (Toyota and Gilroy, 2013. Am. J. Bot. 100 (1):111-125). Thus, concentrations of auxin (IAA) and ABA were measured in actively growing shoot tissues from four weeping and four standard 1-year-old greenhouse-grown trees.

Phytohormone analysis was performed using four biological replicates each of weeping and standard trees (Proteomics & Mass Spectrometry Facility at the Danforth Plant Science Center, St. Louis Mo.). 200 mg of fresh tissues was harvested from young actively growing peach shoot tips sampled from four weeping and four non-weeping greenhouse grown trees. All eight samples were analyzed for auxin (IAA), IAA-Asp, abscisic acid (ABA), salicylic acid (SA), jasmonic ccid (JA), oxo-phytodienoic acid (OPDA), and JA-IIe concentrations. No significant differences between standard and weeping peach trees were found for any hormone. These results were consistent with the grafting experiment that weep phenotype is unlikely mediated by a defective mobile molecule such as a phytohormone.

### Example 7. Generating WEEP Silencing Vector and Transgenic Plums

To confirm WEEP function, the expression of the homolog of Ppa013325 was silenced in plum (Prunus domestica) using RNAi-mediated silencing. While peach is not amenable for transformation, the plum (P. domestica) is a transformable species closely related to peach (Hollender, et al., supra; Guseman, et al., 2016., Plant J. 89 (6): 1093-1105; Petri, et al., 2008. Mol. Breed 22:581-591). The 396 bp peach WEEP cds was amplified from RNA from a standard peach with the Superscript® III One-Step RT PRC with Platinum® Taq DNA Polymerase kit (Thermo Fisher Scientific, Waltham, Mass.) and primers PpWEEP-CDS-F-SAL1 (5′-ATGTCGACGGCGTTATGATGAGGGAGAT-3′, forward; SEQ ID NO: 9) and PpWEEP-CDS-R (STP)-Smal (5′-ATCCCGGGTTATGGTTCCAGCTTCAAGGA-3′, reverse; SEQ ID NO: 10). The amplicon (SEQ ID NO: 11) was then cloned into the Invitrogen pCR™8/GW/TOPO® vector before being transferred into the hairpin silencing vector pHellsgate 8 through LR reaction. The expression vector pHellsgate 8 contains the WEEP coding sequence and the reverse complementary sequence thereof, separated by a linker such that a dsRNA is produced by the expression vector pHellsgate 8. The WEEP pHellsgate 8 was subsequently transformed into Agrobacterium tumefaciens GV3101 and then into the plum cultivar Stanley using previously described hypocotyl slice tissue culture methods (Petri, et al., 2008. Mol. Breed. 22:581-591; Hollender, et al., supra). The transformed plum cells were selected for those cells that contain the expression vector and produce the dsRNA. The selected transformed plum cells then were induced to grow into seedlings and saplings.

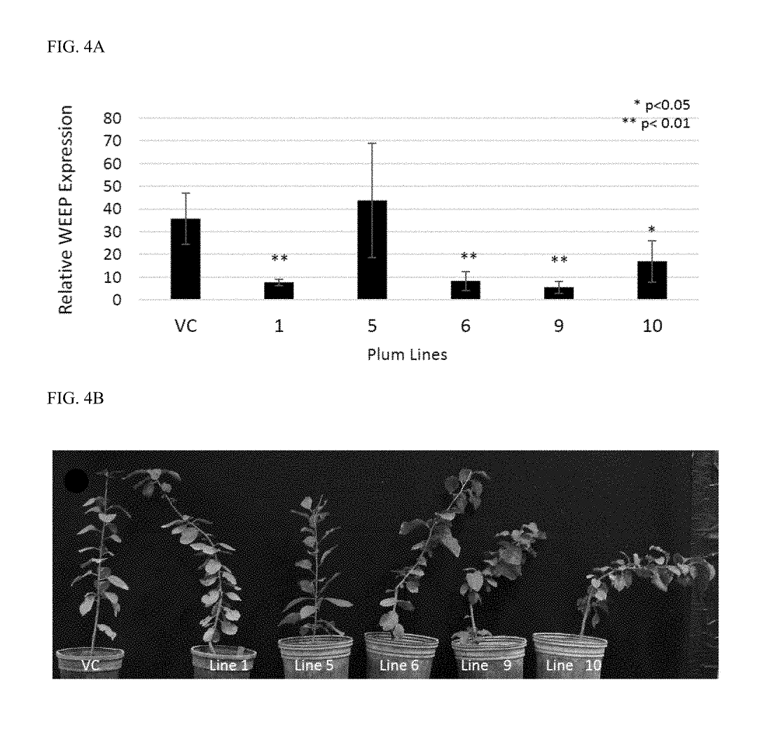

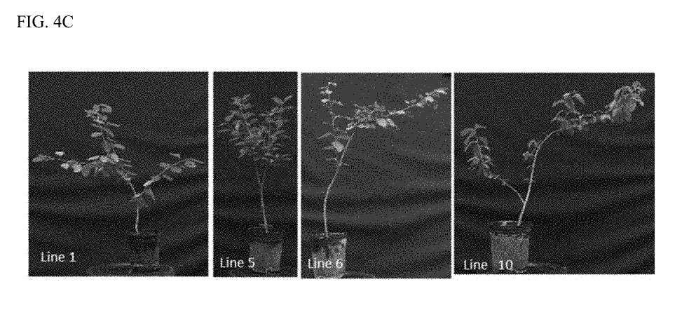

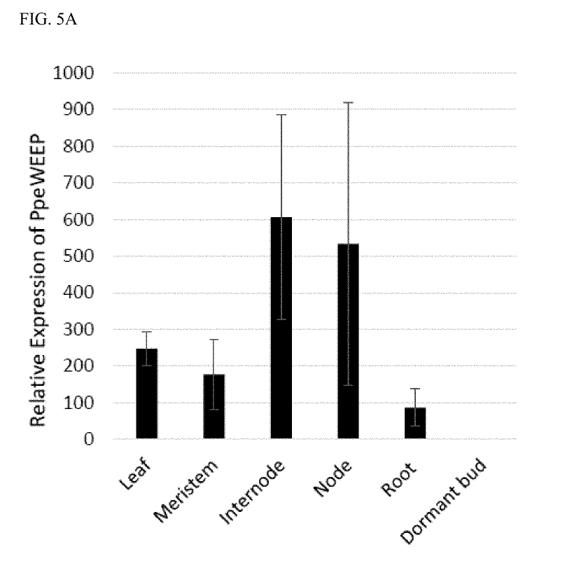

The relative expression of WEEP was examined in genetically altered plum lines 1, 5, 6, 9, and 10. RNA was extracted from actively growing shoot apical and young leaf tissues from greenhouse grown genetically altered plum lines 1, 5, 6, 9 and 10, using the following protocol. Total RNA was extracted from frozen tissue using an E.Z.N.A. SQ Total RNA Kit (Omega Bio-tek Inc.), according to the manufacturer's instructions, with the exception that 2% polyvinylpyrrolidone (PVP) was added to the RCL buffer. RNA was extracted from young leaf and apical meristem tissue from transgenic and control plums. Also, RNA Clean & Concentrate™ (Zymo Research, Irvine, Calif.) was used for RNA purification instead of phenol/chloroform. WEEP expression levels were determined by quantitative PCR (qPCR) using SuperScript™ III Platinum™ SYBR™ Green One-Step qRT-PCR with Rox (Thermo Fischer Scientific, Waltham, Mass.) and run on an AB17900 (Thermo Fischer Scientific, Waltham, Mass.). 50 ng total RNA were used for each reaction. The primers for amplifying native gene expression in plum were Ppweep qPCR UTR-F (5′-TGCCTAGAGAACAGAGTAGGAAAG-3′, forward; SEQ ID NO: 12) and Ppweep qPCR UTR-R (5′-GACCAGCGATAGATACATTAAAGGC-3′, reverse; SEQ ID NO: 13). Relative expression values were determined based on standard curves made from known amounts of total RNA from standard plum trees. For expression in the transgenic plums, between three and six biological replicates were tested, each with three technical replicates. As shown in FIG. 4A, compared to an empty vector negative control transformation, WEEP expression was strongly reduced in genetically altered plum lines 1, 6, and 9 and reduced in genetically altered plum line 10. In contrast, genetically altered plum line 5 had higher WEEP expression than the negative control plum line.

Shoots of RNAi-silenced plum lines with significant reductions in WEEP expression (lines 1, 6, 9, and 10) all had non-vertical outward, downward, and/or curved branch orientations when compared to empty vector transformed controls. Further, an RNAi transformed plum line with no reduction of expression (line 5) was phenotypically normal. See FIG. 4B. The non-vertical growth phenotypes became more pronounced by the end of their second growing season for lines 1, 6, and 10 (FIG. 4C).

### Example 8. Use of CRISPR/Cas9 to Generate Genetically Altered Prunus persica

To generate a Prunus persica with a mutation within WEEP (Ppe013325) so that a non-functional WEEP protein is produced and which generates a weeping phenotype, the CRISPR-Cas9 system is used to create frame shift mutations in WEEP. CRISPR constructs are generated to target the 1^{st} exon in WEEP (Ppe013325) resulting in frame shift or non-sense mutations that disrupt or prematurely terminate protein translation. The target sequence for the 1^{st} exon is 5′-AAGAAATCAAGAGGCTCTGG-3′ (located on the minus strand; SEQ ID NO: 16). To generate the CRISPR construct targeting the 1^{st} exon of the Prunus persica WEEP gene (Ppe013325), primers 5′-ATTGAAGAAATCAAGAGGCTCTGG-3′ (SEQ ID NO: 18) and 5′-AAACCCAGAGCCTCTTGATTTCTT-3′ (SEQ ID NO: 19) are annealed to generate a single guide RNA (sgRNA) insert fragment 5′-ATTGAAGAAATCAAGAGGCTCTGGGTTT-3′ (SEQ ID NO: 17). The sgRNA insert fragment (SEQ ID NO: 17) contains overhangs (the underlined nucleotides at the 5′ and 3′ end) that facilitate ligation of the sgRNA insert with Bsal digested T-DNA vector (such as pHEE401 E), which contains an egg cell-specific promoter (Wang, et al., Genome Biol. 16:144 (2015)). The resulting constructs are transformed into Agrobacterium tumefaciens strain GV3101 and used for Prunus transformation to facilitate CRISPR gene editing at the target site in WEEP (Ppe013325) as described supra. Transformed plants are selected on MS plates as previously described. To identify genetically altered plants with mutations, the CDS region in transformed plants are amplified using forward primer PpWEEP-CDS-F-SAL1 (SEQ ID NO: 9) and reverse primers PpWEEP-CDS-R (STP)-Smal (SEQ ID NO: 10) to amplify the region including the 1st exon. Amplicons are sequenced using standard Sanger sequencing with either forward primer PpWEEP-CDS-F-SAL1 (SEQ ID NO: 9) or reverse primers PpWEEP-CDS-R (STP)-Smal (SEQ ID NO: 10). Selected genetically altered plants are then grown and propagated.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

We, the inventors, claim:

1. A dsRNA comprising at least any contiguous 19 nucleotides of SEQ ID NO: 4, a linker, and a sequence complementary to said at least any contiguous 19 nucleotides of SEQ ID NO: 4, wherein said dsRNA induces RNAi of WEEP in a eudicot plant.

2. An expression vector comprising a heterologous promoter operably linked to a polynucleotide encoding said dsRNA of claim 1.

3. A transformed plant cell comprising said expression vector of claim 2.

4. A genetically altered eudicot plant, part thereof, and progeny thereof having a weeping phenotype compared to a non-weeping phenotype of a wild-type eudicot plant, said genetically altered eudicot plant, parts, and progeny thereof comprise a genetic alteration that reduces amount of functional WEEP protein within said genetically altered plant, part, and progeny thereof compared to amount of functional WEEP protein produced by said wild-type eudicot plant, wherein said reduced amount of said functional WEEP protein in said genetically altered eudicot plant, part, and progeny thereof causes said genetically altered eudicot plant, part, and progeny thereof to have said weeping phenotype compared to said wild-type eudicot plant's phenotype.

5. The genetically altered eudicot plant, part, and progeny thereof of claim 4, wherein said genetic alteration is selected from the group consisting of (i) a null mutation in WEEP wherein said null mutation in WEEP is not the sequence of SEQ ID NO: 36; (ii) a deletion of WEEP wherein said deletion of WEEP is not the sequence of SEQ ID NO: 36; and (iii) an expression vector comprising a heterologous promoter operably linked to a polynucleotide encoding at least any contiguous 19 nucleotides of WEEP, a linker, and sequence complementary to said at least any contiguous 19 nucleotides of WEEP, wherein said expression vector produces a WEEP dsRNA, and wherein said WEEP dsRNA reduces production of functional WEEP in said genetically altered eudicot plant, part, and progeny thereof compared to amount of functional WEEP produced in said wild-type eudicot plant.

6. The genetically altered eudicot plant, part, and progeny thereof of claim 5, wherein said null mutation in WEEP alters WEEPs coding sequence so that a non-functional WEEP is produced by said genetically altered eudicot plant, part, and progeny thereof.

7. The genetically altered eudicot plant, part, and progeny thereof of claim 5, wherein said deletion mutation either removes ATG codon at nucleotides 1-3 of SEQ ID NO: 2 or creates a frame shift in said WEEP DNA sequence.

8. The genetically altered eudicot plant, part, and progeny thereof of claim 5, wherein said polynucleotide encoding said WEEP dsRNA comprises a sequence of at least 19 contiguous nucleotides of a gene encoding a WEEP protein that has an amino acid sequence of 95% or greater identity to SEQ ID NO: 35.

9. The genetically altered eudicot plant, part, and progeny thereof of claim 8, wherein said WEEP protein has an amino acid sequence selected from the group consisting of SEQ ID NO: 20, 21, 22, 23, 24, 25, and 26.

10. The genetically altered eudicot plant, part, and progeny thereof of claim 8, wherein said gene encoding said WEEP protein has a DNA sequence selected from the group consisting of SEQ ID NO: 27, 28, 29, 30, 31, 32, and 33.

11. The genetically altered eudicot plant, part, and progeny thereof of claim 5, where said polynucleotide encoding said WEEP dsRNA comprises SEQ ID NO: 4, a linker, and a sequence complementary to SEQ ID NO: 4.

12. A method for generating a weeping phenotype in a genetically altered eudicot plant compared a wild-type eudicot plant's non-weeping phenotype, said method comprising (i) creating a genetic alteration in said wild-type eudicot plant cell's genome to generate a transformed eudicot cell, (ii) selecting said transformed eudicot cell that expresses said genetic alteration to produce a selected genetically altered eudicot cell, and (iii) inducing said selected genetically altered eudicot cell to grow into a genetically altered eudicot plant that expresses said alteration, wherein said alteration causes a reduced amount of functional WEEP to be produced by said genetically altered eudicot plant compared to amount of functional WEEP produced by said wild-type eudicot plant, wherein said reduced amount of functional WEEP causes said weeping phenotype in said genetically altered eudicot plant compared to said non-weeping phenotype in said wild-type eudicot plant.

13. The method of claim 12, wherein said step of creating said genetic alteration in said wild-type eudicot cell's genome comprises transforming said wild-type eudicot plant cell with an expression vector, wherein said expression vector comprises a heterologous promoter operably linked to a polynucleotide encoding a dsRNA, wherein said polynucleotide comprises any contiguous 19 nucleotides of WEEP, a linker, and a sequence complementary to said at least any contiguous 19 nucleotides of WEEP to generate said genetically altered eudicot plant cell, and wherein said genetic alteration produces said dsRNA.

14. The method of claim 13, wherein said polynucleotide encoding said dsRNA contains at least 19 contiguous nucleotides from a DNA sequence that encodes a protein that has 95% or greater identity to SEQ ID NO: 35.

15. The method of claim 14, wherein said protein that has 95% or greater identity to SEQ ID NO: 35 is selected from the group consisting of SEQ ID NO: 20, 21, 22, 23, 24, 25, and 26.

16. The method of claim 13, wherein said dsRNA contains a sense sequence of at least 19 contiguous nucleotides from the group consisting of SEQ ID NO: 27, 28, 29, 30, 31, 32, and 33.

17. The method of claim 13, wherein SEQ ID NO: 4 encodes said dsRNA's antisense sequence.

18. The method of claim 12, wherein said step of creating said genetic alteration in said wild-type eudicot cell's genome comprises inducing a targeted cleavage event in said wild-type eudicot plant cell to generate a genetically altered WEEP, wherein said genetically altered WEEP encodes an altered WEEP having reduced functionality compared to wild-type WEEP's functionality, and wherein said genetic alteration causes production of said altered WEEP.

19. The method of claim 18, wherein said inducing a targeted cleavage event further comprises transforming said wild-type plant eudicot cell with an expression vector encoding an RNA-guided DNA endonuclease and a polynucleotide encoding a sgRNA that causes said genetic alteration in said WEEP.

20. The method of claim 19, wherein said WEEP comprises a DNA sequence that encodes a WEEP protein that has 95% or great identity to SEQ ID NO: 35.

21. The method of claim 20, wherein said WEEP that has 95% or greater identity to SEQ ID NO: 35 is selected from the group consisting of SEQ ID NO: 20, 21, 22, 23, 24, 25, and 26.

22. The method of claim 19, wherein said a polynucleotide encoding a sgRNA comprising 20 contiguous nucleotides from SEQ ID NO: 27, 28, 29, 30, 31, 32, and 33.

23. The method of claim 22, wherein said sgRNA is selected from the group consisting of SEQ ID NO: 16, 37, 38, 39, 40, and 41.

24. A genetically altered eudicot, part thereof, and progeny thereof, having a weeping phenotype, wherein said genetically altered eudicot, part, and progeny thereof, is produced by the method of claim 12, and wherein said genetically altered eudicot produces reduced amount of functional WEEP compared to amount of functional WEEP produced by wild-type eudicot.
